SEQUENCE LISTING (34 sequences)

SEQ ID NO: 1 - DNA, 32 bp, Artificial sequence
  Oligonucleotide primer,  XmaI-pPlas.c
agttccccgggctggtatatttatatgttgtc

SEQ ID NO: 2 - DNA, 46 bp, Artificial sequence
  Oligonucleotide primer, SacI-ATG-pPlas.r
aatagagctccattttctctcaagatgattaattaattaattagtc

SEQ ID NO: 3 - DNA, 46 bp, Artificial sequence
  Oligonucleotide primer, SacI-PlasTer.c
aatagagctcgttaaaatgcttcttcgtctcctatttataatatgg

SEQ ID NO: 4 - DNA, 48 bp, Artificial sequence
  Oligonucleotide primer, EcoRI-PlasTer.r
ttacgaattctccttcctaattggtgtactatcatttatcaaagggga

SEQ ID NO: 5 - DNA, 25 bp, Artificial sequence
  Olilgonucleotide primer, Plasto-443c
gtattagtaattagaatttggtgtc

SEQ ID NO: 6 - DNA, 34 bp, Artificial sequence
  Oligonucleotide primer, Plas+LC-C51.r
atctgaggtgtgaaaaccattttctctcaagatg

SEQ ID NO: 7 - DNA, 26 bp, Artificial sequence
  Oligonucleotide primer, LC-C51.c
atggttttcacacctcagatacttgg

SEQ ID NO: 8 - DNA, 39 bp, Artificial sequence
  Oligonucleotide primer, LC-C51XhoSac.r
atatgagctcctcgagctaacactcattcctgttgaagc

SEQ ID NO: 9 - DNA, 35 bp, Artificial sequence
  Oligonucleotide primer, Plas+HC-C51.r
caaggtccacacccaagccattttctctcaagatg

SEQ ID NO: 10 - DNA, 22 bp, Artificial sequence
  Oligonucleotide primer, HC-C51.c
atggcttgggtgtggaccttgc

SEQ ID NO: 11 - DNA, 36 bp, Artificial sequence
  Oligonucleotide primer, HC-C51XhoSac.r
ataagagctcctcgagtcatttaccaggagagtggg

SEQ ID NO: 12 - DNA, 42 bp, Artificial sequence
  Oligonucleotide primer, HC-C51KDEL(SacI).r
ataagagctctcaaagttcatcctttttaccaggagagtggg

SEQ ID NO: 13 - skipped

SEQ ID NO: 14 - DNA, 1227 bp, Homo sapien
  Nucleotide sequence for hGalT
atgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggggctggatgtacagagatacccattgtatacccaaatcacagtggacatcgggacaccgagcgattacaaggatgacgatgacaagatcgattag

SEQ ID NO: 15 - protein, 408 aa, Homo sapien
  Amino acid sequence for hGalT
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQGLDVQRYPLYTQITVDIGTPSDYKDDDDKID

SEQ ID NO: 16 - DNA, 42 bp, Artificial sequence
  Primer Tev+HC-C51.2
caaggtccacacccaagccattgctatcgttcgtaaatggtg

SEQ ID NO: 17 - DNA, 1095 bp, Artificial sequence
  Nucleotide sequence  of GNT1-GalT
atgagagggtacaagttttgctgtgatttccggtacctcctcatcttggctgctgtcgccttcatctacatacagatgcggctttttgcgacacagtcagaatatgcagatcgccttgctgctgcaattgaagcagaaaatcactgtacaagtcagaccagattgcttattgaccagattagccagcagcaaggaagaatagttgctcttgaagaacaaatgaagcgtcagactagtgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggggctggatgtacagagatacccattgtatacccaaatcacagtggacatcgggacaccgagcgattacaaggatgacgatgacaagatcgattag

SEQ ID NO: 18 - protein, 364 aa, Artificial sequence
  Amino acid sequence of GNT-GalT
MRGYKFCCDFRYLLILAAVAFIYIQMRLFATQSEYADRLAAAIEAENHCTSQTRLLIDQISQQQGRIVALEEQMKRQTSALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQGLDVQRYPLYTQITVDIGTPSDYKDDDDKID

SEQ ID NO: 19 - DNA, 1002 bp, Artificial sequence
  Nucleotide sequence for plastocyanin promoter and 5′ UTR
agaggtaccccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatg

SEQ ID NO: 20 - DNA, 399 bp, Artificial sequence
  Nucleotide sequence for the plastocyanin 3′ UTR and terminator
taagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtaca

SEQ ID NO: 21 - DNA, 54 bp, Artificial sequence
  Nucleotide sequence of a CTS domain of N-acetylglucosamine transferase of GNT1
tacctcctcatcttggctgctgtcgccttcatctacatacagatgcggcttttt

SEQ ID NO: 22 - protein, 18 aa, Artificial sequence
  Amino acid of the CTS domain of  GNT1
YLLILAAVAFIYIQMRLF

SEQ ID NO: 23 - DNA, 37 bp, Artificial sequence
  Oligonucleotide primer, XhoTEV.c
tttggagaggacctcgagaaataacaaatctcaacac

SEQ ID NO: 24 - DNA, 40 bp, Artificial sequence
  Oligonucleotide primer, TEV+LC-C5-1.r
atctgaggtgtgaaaccattgctatcgttcgtaaatggtg

SEQ ID NO: 25 - DNA, 26 bp, Artificial sequence
  Oligonucleotide primer, LC-C5-1.c
atggttttcacacctcagatacttgg

SEQ ID NO: 26 - DNA, 39 bp, Artificial sequence
  Oligonucleotide primer, LC-C5-1SphSac.r
atatgagctgcgatgcctaacactcattcctgttgaagc

SEQ ID NO: 27 - DNA, 38 bp, Artificial sequence
  Oligonucleotide primer, FgalT
gactctagagcgggaagatgaggcttcgggagccgctc

SEQ ID NO: 28 - DNA, 58 bp, Artificial sequence
  Oligonucleotide primer, RgalTFlagStu
aaggcctacgctacttgtcatcgtcatctttgtagtcgcacggtgtcccgaagtccac

SEQ ID NO: 29 - DNA, 35 bp, Artificial sequence
  Oligonucleotide primer, FGNT
atcgaaatcgcacgatgagagggaacaagttttgc

SEQ ID NO: 30 - DNA, 35 bp, Artificial sequence
  Oligonucleotide primer, RGNTSpe
cgggatccactagtctgacgcttcatttgttcttc

SEQ ID NO: 31 - DNA, 29 bp, Artificial sequence
  Oligonucleotide primer, FgalTSpe
ggactagtgcactgtcgctgcccgcctgc

SEQ ID NO: 32 - DNA, 36 bp, Artificial sequence
  Primer HC-C51SphSac.r
ataagagctcgcatgctcatttaccaggagagtggg

SEQ ID NO: 33 - DNA, 773 bp, Artificial sequence
  2X35S promoter of intermediary plasmid, including restriction enzyme sites
aagcttgcatgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagttgattgatgtgatatctccactgacgtaagggatgacgcacaatcccactatccttcgcaagacccttcctctatataaggaagttcatttcatttggagaggacctcgag

SEQ ID NO: 34 - DNA, 277 bp, Artificial sequence
  NOS terminator of intermediary plasmid with restriction enzyme sites included
gagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattc

(19) United States
(12) Patent Application Publication
(10) Pub. No.: US 2010/0251417 A1
(43) Pub. Date: Sep. 30, 2010

(54) PROTEIN PRODUCTION IN PLANTS

(76) Inventors: Marc-Andre D'Aoust, Quebec (CA); Julie Belles-Isles, Quebec (CA); Nicole Bechtold, Quebec (CA); Michele Martel, Quebec (CA); Pierre-Olivier Lavoie, Quebec (CA); Louis-Philippe Vezina, Neuville (CA)

Correspondence Address: QUARLES & BRADY LLP, 411 E. WISCONSIN AVE., SUITE 2040, MILWAUKEE, WI, 53202-4497, US

(21) Appl. No.: 12/663,987

(22) PCT Filed: Jun. 13, 2008
(86) PCT No.: PCT/CA08/01146

§ 371 Date: May 12, 2010

Related U.S. Application Data
(60) Provisional application No. 60/944,370, filed on Jun. 15, 2007.

(51) Int. Cl. C12N 15/82

(52) U.S. Cl. 800/278

(57) ABSTRACT

A method for synthesizing a protein of interest within a plant or a portion of a plant is provided. The method involves introducing one or more than one nucleic acid sequence encoding a protein of interest operatively linked with a regulatory region obtained from a photosynthetic gene that is active in the plant, in a transient manner. The plant is then maintained under conditions that permit the nucleic acid sequence encoding the protein of interest to be expressed in the plant or the portion of the plant. The plant may be pruned prior to the introducing one or more than one nucleic acid sequence.

### FIELD OF INVENTION

The present invention relates to methods of producing protein in plants. The present invention also provides nucleotide sequences that may be used for producing proteins in plants.

### BACKGROUND OF THE INVENTION

Immunoglobulins (IgGs) are complex heteromultimeric proteins with characteristic affinity for specific antigenic counterparts of various natures. Today, routine isolation of IgG-producing cell lines, and the advent of technologies for IgG directed evolution and molecular engineering have profoundly impacted their evolution as biotherapeutics and in the general life science market. Therapeutic monoclonal IgG (monoclonal antibodies, mAbs) dominate the current market of new anti-inflammatory and anti-cancer drugs and hundreds of new candidates are currently under research and clinical development for improved or novel applications. The annual market demand for mAbs ranges from a few grams (diagnostics), a few kilograms (anti-toxin) to up to one or several hundreds of kilograms (bio-defense, anti-cancer, anti-infectious, anti-inflammatory).

Although CHO cell culture is still their preferred production host at commercial scale, it is generally accepted that for mAbs to reach their full impact on the life science market, alternative production systems have to be developed, as the facilities required for these cultures are not easily modulated in scale, their building and maintenance costs are extremely high and steadily increasing, and their validation under GMP still requires an average of three years following construction. Even at the early development stages, the selection of CHO cell lines with acceptable yields and productivity remains a costly and long process. New production systems that would decrease the upstream costs (higher yields, simpler technologies and infrastructures), have shorter lead time, be more flexible in capacity, while meeting the current reproducibility, quality and safety features of current cell culture systems are likely to have a significant impact on the development of mAbs and vaccines for the life science market, at every development stages.

Plants are suitable hosts for the production of mAbs and several other proteins which have current applications in life sciences (see Ko and Koprowski 2005; Ma et al., 2005; Yusibov et al., 2006 for recent reviews). MAbs have been produced in stable transgenic plant lines at yields up to 200 mg/kg fresh weight (FW), and through transient expression at rates of up to 20 mg/kg FW (Kathuria, 2002). Giritch et al. (2006) report expression levels of 200-300 mg/kg of leaf weight for an IgG, with one cited maximum of 500 mg/kg through the use of a multi-virus based transient expression system.

Many of the transient systems described to date for the synthesis of mAbs (e.g. Kapila et al. 1997; Vaquero et al. 1999, Rodriguez et al. 2004) may involve complex procedures, result in low levels of accumulation of the product, or both. Alternate methods that result in high yields of proteins are desired.

### SUMMARY OF THE INVENTION

The present invention relates to methods of producing protein in plants. The present invention also provides nucleotide sequences that may be used for producing proteins in plants.

It is an object of the invention to provide an improved method for producing protein in plants.

The present invention provides a method (A) for synthesizing a protein of interest within a plant or a portion of a plant comprising,

i) pruning the plant or portion of the plant to produce a pruned plant or portion of the plant,

ii) introducing one or more than one nucleic acid sequence encoding a protein of interest operatively linked with a regulatory region that is active in the plant, into the pruned plant or portion of the plant in a transient manner, and

iii) maintaining the pruned plant or portion of the plant under conditions that permit the nucleic acid sequence encoding the protein of interest to be expressed in the plant or a portion of the plant.

The protein of interest may be an antibody, an antigen, a vaccine or an enzyme.

The present invention also pertains to the methods as described above wherein, in the step of introducing (step ii), two or more than two nucleic acid sequences may be introduced within the plant. Furthermore, one of the two or more than two nucleic acid sequences may encode a suppressor of silencing. For example, the suppressor of silencing may be HcPro, TEV-p1/HC-Pro, BYV-p21, TBSV p19, TCV-CP, CMV-2b, PVX-p25, PVM-p11, PVS-p11, BScV-p16, CTV-p23, GLRaV-2 p24, GBV-p14, HLV-p10, GCLV-p16, or GVA-p10.

The present invention includes the method described above wherein, in the step of introducing (step ii), the one or more than one nucleic acid sequence may be introduced into the pruned plant or portion of the plant using agrobacterium. The agrobacterium may be introduced into the pruned plant or portion of the plant under vacuum or by using a syringe. Furthermore, in the step of introducing (step ii) as described above, the regulatory region includes a promoter obtained from a photosynthetic gene. For example, the regulatory region may include a plastocycanin promoter, plastocyanin a 3′UTR transcription termination sequence, or both a plastocycanin promoter and plastocyanin a 3′UTR transcription termination sequence.

The present invention also pertains to a method (B) for synthesizing a protein of interest within a plant or a portion of a plant comprising,

i) introducing one or more than one nucleic acid sequence encoding a protein of interest operatively linked with a regulatory region obtained from a photosynthetic gene that is active in the plant or portion of the plant in a transient manner, and

ii) maintaining the plant or portion of the plant under conditions that permit the nucleic acid sequence encoding the protein of interest to be expressed in the plant or a portion of the plant.

The protein of interest may be an antibody, an antigen, a vaccine or an enzyme.

The present invention also pertains to the method (B) as described above wherein, in the step of introducing (step i), two or more than two nucleic acid sequences are be introduced within the plant. Furthermore, one of the two or more than two nucleic acid sequences may encode a suppressor of silencing. For example, the suppressor of silencing may be HcPro, TEV-p1/HC-Pro, BYV-p21, TBSV p19, TCV-CP, CMV-2b, PVX-p25, PVM-p11, PVS-p11, BScV-p16, CTV-p23, GLRaV-2 p24, GBV-p14, HLV-p10, GCLV-p16, or GVA-p10.

The present invention includes the method (B) described above wherein, in the step of introducing (step i), the one or more than one nucleic acid sequence may be introduced into the pruned plant or portion of the plant using agrobacterium. The agrobacterium may be introduced into the pruned plant or portion of the plant under vacuum or by using a syringe. Furthermore, in the step of introducing (step ii) as described above, the regulatory region includes a promoter obtained from a photosynthetic gene. For example, the regulatory region may include a plastocycanin promoter, plastocyanin a 3′UTR transcription termination sequence, or both a plastocycanin promoter and plastocyanin a 3′UTR transcription termination sequence.

The present invention also provides a method (Method C) for synthesizing a protein of interest within a plant or a portion of a plant comprising,

i) pruning the plant or portion of the plant to produce a pruned plant or portion of the plant,

ii) introducing one or more than one nucleic acid sequence encoding a protein of interest operatively linked with a regulatory region obtained from a photosynthetic gene that is active in the plant or portion of the plant, in a transient manner, and

iii) maintaining the pruned plant or portion of the plant under conditions that permit the nucleic acid sequence encoding the protein of interest to be expressed in the plant or a portion of the plant.

The protein of interest may be an antibody, an antigen, a vaccine or an enzyme.

The present invention also pertains to the method (C) as described above wherein, in the step of introducing (step ii), two or more than two nucleic acid sequences are be introduced within the plant. Furthermore, one of the two or more than two nucleic acid sequences may encode a suppressor of silencing. For example, the suppressor of silencing may be HcPro, TEV-p1/HC-Pro, BYV-p21, TBSV p19, TCV-CP, CMV-2b, PVX-p25, PVM-p11, PVS-p11, BScV-p16, CTV-p23, GLRaV-2 p24, GBV-p14, HLV-p10, GCLV-p16, or GVA-p10.

The present invention includes the method (C) described above wherein, in the step of introducing (step ii), the one or more than one nucleic acid sequence may be introduced into the pruned plant or portion of the plant using agrobacterium. The agrobacterium may be introduced into the pruned plant or portion of the plant under vacuum or by using a syringe. Furthermore, in the step of introducing (step ii) as described above, the regulatory region includes a promoter obtained from a photosynthetic gene. For example, the regulatory region may include a plastocycanin promoter, plastocyanin a 3′UTR transcription termination sequence, or both a plastocycanin promoter and plastocyanin a 3′UTR transcription termination sequence.

The present invention provides a simplified plant expression system for driving the expression of a protein of interest in a plant using a transient expression system. According to the methods described herein a protein of interest may be produced in high yield. The transient co-expression system described herein avoids lengthy production times, and the selection process of elite mutant or glyco-engineered transgenic lines and their subsequent use as parental lines as described in the prior art (e.g. Bakker, 2005). It also avoids the concurrent problems often encountered with mutant or glyco-engineered plants, in terms of productivity, pollen production, seed set (Bakker et al 2005) and viability (Boisson et al., 2005). The transient expression system described herein yields expression levels reaching 1.5 g of high quality antibody per kilogram of leaf fresh weight, exceeding the accumulation level reported for any antibody in plants with other expression systems, including multi-virus based systems and transgenic plants.

As described herein, pruning plants before infiltration of the desired nucleic acid construct was observed to increase expression level (as a % of total synthesized protein) and yield (mg of protein/kg of fresh weight). This was observed using several methods of infiltration including but not limited to syringe-infiltration or vacuum-infiltration. A variety of methods of pruning, for example but not limited to mechanical pruning, or chemical pruning, increased expression levels and protein yield.

The use of a regulatory region from a photosynthetic gene, for example but not limited to that obtained from the gene encoding the large or small subunit of ribulose 1,5-bisphosphate carboxylase/oxygenase (Rubisco) or plastocyanin, or the use of a regulatory region from a photosynthetic gene was found to increase expression levels and yield. Furthermore, the use of a regulatory region from a photosynthetic gene in combination with pruning was found to increase expression levels and yield.

Infiltration technology allows for the production of grams of this antibody per day within a small pilot unit, which permits the use of such transient expression system for the production of materials for clinical trials within extremely short time frames and for the supply of a licensed product with a market size up to kilograms per year. High quality antibodies were obtained from infiltrated leaves after a single affinity chromatographic step.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1A shows examples of expression cassettes assembled for expression of several proteins. R612 comprises a nucleotide sequence encoding C5-1 LC and C5-1 HC each under the control of a plastocyanin promoter and 5′UTR, and a plastocyanin terminator. R610 comprises a nucleotide sequence encoding C5-1 LC and C5-1 HC-KDEL each under the control of a plastocyanin promoter and 5′UTR, and a plastocyanin terminator. R514, comprises a nucleotide sequence encoding C5-1 LC and C5-1 HC. C5-1 LC: C5-1 light chain coding sequence each under the control of 2X35S promoter the tobacco etch virus (TEV) leader sequence and a NOS terminator; C5-1 LC: C5-1 light chain coding sequence; C5-1 HC: C5-1 heavy chain coding sequence. 935 comprises a nucleotide sequence encoding a human IgG-LC and a human IgG-HC each under the control of a plastocyanin promoter and 5′UTR, and a plastocyanin terminator. 312 comprises a nucleotide sequence encoding a flu antigen under the control of a plastocyanin promoter and 5′UTR, and a plastocyanin terminator. FIG. 1B shows the nucleotide sequence for the plastocyanin promoter and 5′ UTR (SEQ ID NO:19), the transcription start site is shown in bold, and the translation start codon is underlined. FIG. 1C shows the nucleotide sequence for the plastocyanin 3′ UTR and terminator (SEQ ID NO:20), the stop codon is underlined. FIG. 1D shows 2X35S (SEQ ID NO:33) and NOS (SEQ ID NO:34) sequences in the intermediary plasmid used for R512 and R513 assembly. NOS terminator (SEQ ID NO:34) is in italics; 2X35S promoter is bolded (SEQ ID NO:33). Restriction enzyme sites are underlined.

FIG. 2 shows accumulation of the C5-1 antibody in leaves of Nicotiana benthamiana infiltrated with various expression cassettes. FIG. 2A shows accumulation of the C5-1 antibody produced following syringe infiltration of R514 (a 35S based expression cassette), R610 and R612 (plastocyanin based expression cassettes) with or without co-expression of a suppressor of silencing, for example, HcPro. FIG. 2B shows accumulation of the C5-1 antibody using R610 and R612, plastocyanin based expression cassettes, with or without co-expression of a suppressor of silencing (for example HcPro) in vacuum infiltrated or syringe infiltrated leaves. The values presented correspond to the mean accumulation level and standard deviation obtained from the 6 measurements on 3 plants (syringe) or 6 measurements on individual infiltration batches of approximately 12 plants (250 g).

FIG. 3 shows protein blot analysis of C5-1 accumulation in extracts of syringe- and vacuum-infiltrated plants. FIG. 3A shows immunoblotting with a peroxidase-conjugated goat-anti mouse IgG (H+L), on extracts from plants infiltrated with R612 (for secretion, lanes 1) or with R610 (for ER-retention, lanes 2). C₁: 100 ng of commercial murine IgG1 (Sigma M9269), loaded as a control for electrophoretic mobility; C₂: 12 μg of total proteins extracted from mock-infiltrated biomass (empty vector). C₃: 100 ng of commercial murine IgG1 (Sigma M9269) spiked in 12 μg of total protein extracted from mock-infiltrated biomass (empty vector). FIG. 3B shows activity immunoblotting with a peroxidase conjugated human IgG1, on extracts from plants infiltrated with R612 (for secretion, lanes 1) or with R610 (for ER-retention, lanes 2). C₁: 2 μg of control C5-1 purified from hybridoma (Khoudi et al., 1999); C₂: 75 μg of total proteins extracted from mock-infiltrated biomass (empty vector).

FIG. 4 shows an analysis of antibodies purified from plants infiltrated with either R612 (for secretion, lanes 1) or R610 (for ER-retention, lanes 2). FIG. 4A shows SDS-PAGE of crude extracts and purified antibodies was performed in non-reducing conditions. FIG. 4B shows SDS-PAGE of purified antibodies was performed under reducing conditions FIG. 4C shows activity immunoblotting of purified antibodies was performed with a peroxidase conjugated human IgG1 FIG. 4D shows comparison of contaminants in 6 lots of purified C5-1 from different infiltration batches. C: 2.5 μg of commercial murine IgG1 (Sigma M9269), loaded as a control for electrophoretic mobility.

FIG. 5A shows a representation of examples of cassettes assembled for native (R622) and hybrid (R621) versions of galactosyltransferase expression. GNTI-CTS: CTS domain of N-acetylglucos-aminyltransferase I; GalT-CAT: catalytic domain of human β1,4galactosyltransferase; GalT: human β1,4galactosyltransferase. FIG. 5B shows the nucleotide sequence (SEQ ID NO: 14) for GalT (UDP-Gal:betaGlcNac beta 1,4-galactosyltransferase polypeptide 1, beta-1,4-galactosyltrasnferase I), the ATG start site is underlined; the transmembrane domain is underlined and in italics; the sequence in bold corresponds to the catalytic domain of human beta1,4GalT; the FLAG epitope is in italics. FIG. 5C shows the amino acid sequence (SEQ ID NO: 15) for GalT (UDP-Gal:betaGlcNac beta 1,4-galactosyltransferase polypeptide 1, beta-1,4-galactosyltrasnferase I). The transmembrane domain is underlined and in italics; the sequence in bold corresponds to the catalytic domain of human beta1,4GalT; the FLAG epitope is in italics. FIG. 5D shows the nucleotide sequence (SEQ ID NO: 17) of GNTIGalT, the ATG start site is underlined; the transmembrane domain (CTS) is underlined and in italics; the sequence in bold corresponds to the catalytic domain of human beta1,4GalT; the FLAG epitope is in italics. FIG. 5E shows the amino acid sequence (SEQ ID NO: 18) of GNTIGalT. The transmembrane domain (CTS) is underlined and in italics; the sequence in bold corresponds to the catalytic domain of human beta1,4GalT; the FLAG epitope is in italics. FIG. 5F shows the nucleotide sequence of a CTS domain (cytoplasmic tail, transmembrane domain, stem region) of N-acetylglucosamine transferase (GNT1; SEQ ID NO:21). FIG. 5G shows the amino acid of the CTS (SEQ ID NO:22).

FIG. 6 shows a profile of extracts obtained from plants expressing C5-1 and either stained for protein, or subject to Western analysis. Top panel shows a Commasie stained PAGE gel. Second from the top panel shows affinodetection using Erythrina cristagali agglutinin (ECA) which specifically binds β1,4galactose. Third panel from the top shows Western blot analysis using anti-α1,3fucose antibodies. Bottom panel shows Western blot analysis using anti-β1,2xylose specific antibodies. R612: C5-1 expressed alone; R612+R622: C5-1 co-expressed (co-infiltrated) with GalT; R612+R621: C5-1 co-expressed with GNT1-GalT.

FIG. 7 shows examples of the effect of mechanical or chemical pruning on expression. FIG. 7A shows the effect of pruning, both mechanical pruning, 12 hours prior to infiltration, and chemical pruning, 7 days prior to infiltration, on antigen expression (influenza expression; see FIG. 1, 312) in vacuum agroinfiltrated plants. FIG. 7B shows the effect of mechanical pruning, 12 hours prior to infiltration, on antibody expression (human IgG, see FIG. 1, 935) in vacuum agroinfiltrated plants. FIG. 7C shows the effect of mechanical pruning on antigen (influenza, see FIG. 1, 312) expression in syringe agroinfiltrated plants; Condition 1: control, non pruned plants; Condition 2 mechanically pruned plants.

FIG. 8 shows examples of the effect of the day of pruning (mechanical pruning), 3, 2, or 1 day prior to transformation, or no pruning (control) on antigen accumulation (influenza antigen) in vacuum agroinfiltrated plants.

FIG. 9 shows an example of the combined effect of a suppressor of silencing (HcPro) and pruning (mechanical pruning 12 hours before infiltration) on antibody expression (human IgG, FIG. 1, 935) in vacuum infiltrated plants. Plasto−HcPro−pruning: expression of 935 alone (no pruning, no co-expression of suppressor of silencing); Plasto−HcPro+pruning: mechanical pruning of plants 12 hours before transformation with 935 (no co-expression of suppressor of silencing); Plasto+HcPro−pruning: co-expression of 935 and HcPro (suppressor of silencing; no pruning); Plasto+HcPro+pruning: mechanical pruning 12 hours before coexpression of 935 and HcPro.

### DETAILED DESCRIPTION

The present invention relates to methods of producing protein in plants. The present invention also provides nucleotide sequences that may be used for producing proteins in plants.

A method for synthesizing a protein of interest within a plant, or a portion of a plant, is provided. In its basic form, the method includes introducing one or more than one nucleic acid sequence encoding a protein of interest operatively linked with a regulatory region obtained from a photosynthetic gene that is active in the plant or portion of the plant in a transient manner, and maintaining the plant, or a portion of the plant, under conditions that permit the nucleic acid sequence encoding the protein of interest to be expressed in the plant or a portion of the plant.

The method may further comprises, first pruning the plant or portion of the plant prior to introducing the one or more than one nucleic acid sequence encoding the protein of interest. In this method, after the plant or a portion of the plant has been pruned one or more than one nucleic acid sequence encoding a protein of interest operatively linked with a regulatory region that is active in the plant, is introduced into the pruned plant or portion of the plant in a transient manner. The plant or portion of the plant is then maintained under conditions that permit the nucleic acid sequence encoding the protein of interest to be expressed in the plant or a portion of the plant.

Using this method high yields of the protein of interest have been produced, when the production of protein is compared to producing the same protein of interest using a similar transient transformation protocol that either does not include the use of a regulatory region obtained from a photosynthetic gene, a step of pruning, or both the use of a regulatory region obtained from a photosynthetic gene combined with a step of pruning.

Promoters used in expression cassettes designed for use in stable transgenic expression systems have been found to have low efficiency when used in transient expression systems (Giritch et a. 2006, Fisher, 1999a). Giritch et al (12206) show that using co-expression of different provectors (one based on TMV and the other on PVX) for each IgG subunit, together with one recombinase and two viral replicases were they able to attain expression levels in the range of 200 mg/kg. As described herein, promoters comprising enhancer sequences with demonstrated efficiency in leaf expression, have been found to be effective in transient expression. A non-limiting example includes the promoter used in regulating plastocyanin expression (Pwee and Gray 1993; which is incorporated herein by reference). Without wishing to be bound by theory, attachment of upstream regulatory elements of a photosynthetic gene by attachment to the nuclear matrix may mediate strong expression (Sandhu et al., 1998; Chua et al., 2003). For example up to −784 from the translation start site of the pea plastocyanin gene may be used mediate strong reporter gene expression.

The use of a regulatory region from a photosynthetic gene, for example but not limited to a plastocyanin regulatory region (U.S. Pat. No. 7,125,978; which is incorporated herein by reference), or a regulatory region obtained from Ribulose 1,5-bisphosphate carboxylase/oxygenase (rubisco; U.S. Pat. No. 4,962,028; which is incorporated herein by reference), chlorophyll a/b binding protein (CAB; Leutwiler et a; 1986; which is incorporated herein by reference), ST-LS1 (associated with the oxygen-evolving complex of photosystem II, Stockhaus et al.1989; which is incorporated herein by reference). may be used in accordance with the present invention.

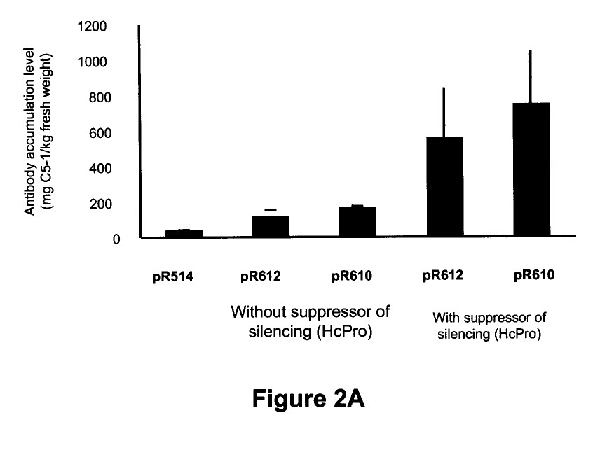

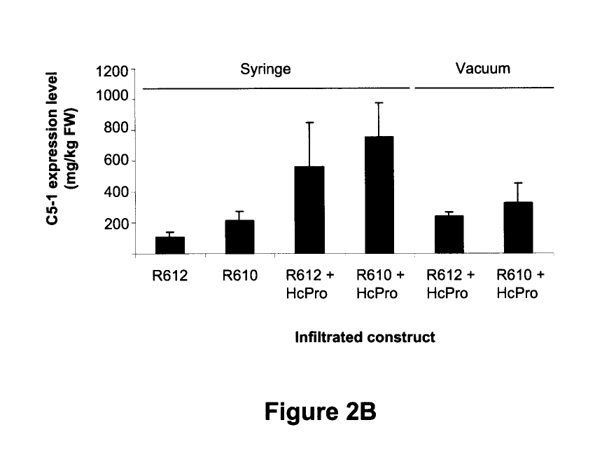

A regulatory region obtained from the gene encoding the large or small subunit of ribulose 1,5-bisphosphate carboxylase/oxygenase (Rubisco) or plastocyanin, or the use of a regulatory region from a photosynthetic gene in combination with pruning was found to increase expression levels and yield. For example, as shown in FIG. 2A levels of expression following infiltration of a coding region of interest driven by the photosynthetic promoter (obtained from plastocycanin; see FIG. 2A, R610, R612) are greater when compared to the same coding region of interest driven by 35S (FIG. 2A, R514).

Therefore, the present invention provides a method for synthesizing a protein of interest within a plant, or a portion of a plant, comprising,

i) introducing one or more than one nucleic acid sequence encoding a protein of interest operatively linked with a regulatory region obtained from a photosynthetic gene that is active in the plant or portion of the plant in a transient manner, and

ii) maintaining the plant, or portion of the plant, under conditions that permit the nucleic acid sequence encoding the protein of interest to be expressed in the plant or a portion of the plant.

The plant, or portion of the plant, may be pruned prior to the step of introducing the one or more than one nucleic acid sequence. Pruning plants before infiltration of the desired nucleic acid construct has been found to increase the level of expression (as a % of total synthesized protein) and yield (mg of protein/kg of fresh weight). This was observed using several methods of infiltration including but not limited to syringe-infiltration or vacuum-infiltration, and a variety of methods of pruning, for example but not limited to mechanical pruning, or chemical pruning. Without wishing to be bound by theory, pruning before infiltration may result in the loss of apical dominance, and may result in a reduction of growth regulator content for example but not limited to gibberellic acid, or ethylene, content. This in turn may stimulate the increase of photosynthetic capacity within a leaf and increase transcription rates of photosynthetic genes. Therefore the use of a regulatory region obtained from a photosynthetic gene may result in higher proteins yields. Furthermore, the combination of the use of a regulatory region obtained from a photosynthetic gene and chemical pruning that reduce the content of growth regulators inhibitors for example, ethylene or gibberellic acid may result in higher proteins yields

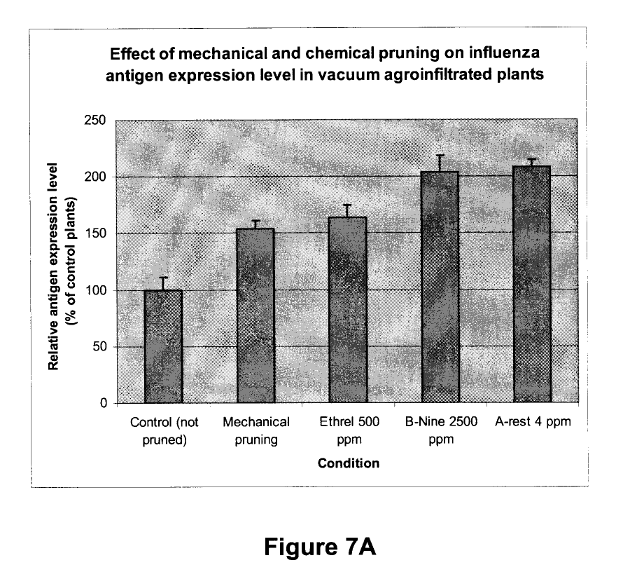

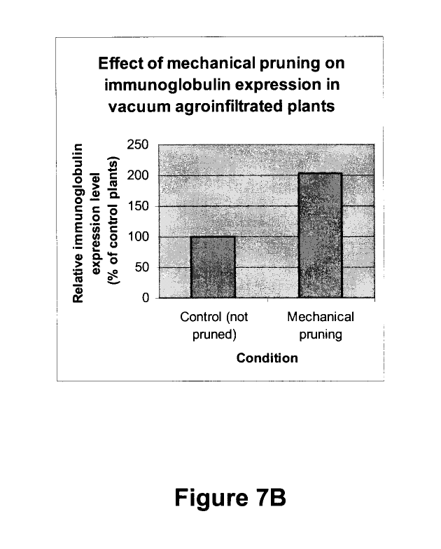

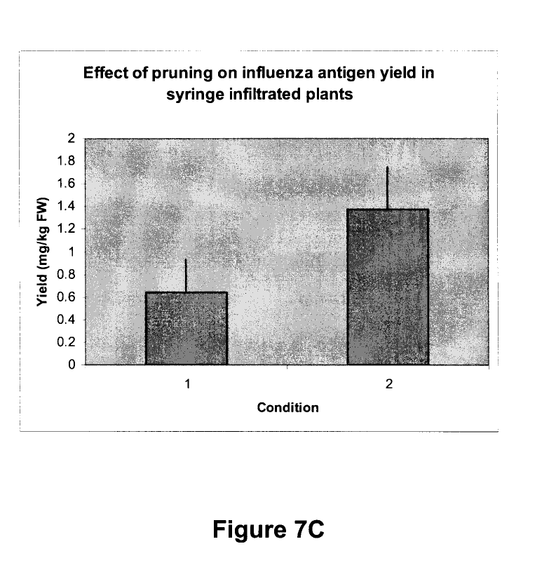

The effect of pruning on the increase in the yield of a protein of interest following the methods as described herein, are observed using either mechanical or chemical pruning methods, and either vacuum or syringe infiltration. The increase in yield due to pruning is observed when the plants are wounded, for example following syringe infiltration (see FIG. 7C). This indicates that the increase in protein expression is not simply a response to plant wounding.

By pruning it is meant the removal of one or more than one axillary bud, one or more than one apical bud, or removal of both one or more than one axillary bud and one or more than one apical bud. Pruning may also include killing, inducing necrosis, or reducing growth of the apical and axillary buds without removing the buds from the plant. By reduction of growth of the bud (or reducing bud growth), it is meant that the bud exhibits a reduction for example in the metabolic activity, or size increase over a defined period of time, of from about 50% to 100%, or any amount therebetween when compared to a bud that has not been treated. Pruning may also be accomplished by applying a chemical compound that reduces apical dominance. If a chemical compound is applied for the purposes of pruning, then the dosages used are typically those as recommended by the manufacturer of the chemical compound.

Pruning, either mechanical or chemical pruning may be carried out from about 20 days prior to infiltration, to about 2 days after infiltration or any time in between, for example 7 days (168 hours) prior to infiltration, to about 2 days (48 hours) after infiltration, or any time in between, for example, from about 48 hours (2 days) prior to infiltration to about 1 day (24 hours) after infiltration, or any time in between, or from 20 days, 19 days, 18, days, 17 days, 16 days, 15 days, 14 days, 13 days, 12 days, 11 days, 10 days, 9 days, 8 days, or 168, 144, 120, 96, 72, 60, 50, 40, 36, 34, 32, 30, 28, 26, 24, 22, 20, 18, 16, 14, 12, 10, 8, 6, 4, 2, 1, 0 hours prior to infiltration, to about 1, 2, 4, 6, 8, 10, 12, 14, 16, 18, 20, 22, 24 hours after infiltration, or any time in between. If pruning is to take place at 72 hours or more prior to infiltration, it is preferred that the pruning method is chemical pruning, as if a mechanical pruning method is used there may be re-growth. If the pruning method is chemical pruning, then a longer period of time prior to infiltration may be used prior to infiltration, for example 2, 3, 4, 5, 6 or 7 days, or any time in between. One of skill can readily determine the appropriate interval prior to pruning.

Pruning can be accomplished by any means that would be known to one of skill in the art and includes, but is not limited to, mechanical removal of the bud, for example but not limited to, cutting, clipping, pinching, compression for example using tongs and the like, localized freezing for example by directing a localized stream of liquid nitrogen to the bud, or surrounding the bud with tongs or other device that has been cooled using an appropriate cold source including liquid nitrogen, dry ice, ethanol-dry ice, ice, and the like, so that the temperature of the bud is reduced so as to reduce growth of the bud, or kill the bud.

Pruning also includes chemical pruning, for example, applying a herbicide (chemical compound; pruning agent) that kills or reduces the growth of the bud, or applying a grow regulator that kills or reduces the growth of the bud. The use of chemical pruning permits an efficient manner of treatment of pruning as plants can be readily treated by spraying, misting, soaking, the chemical compound on the plant, or dipping the plants into a solution comprising the chemical compound. Plants may be treated once prior to the step of infiltration, or treated more than once prior to the step of infiltration. Examples of chemical compounds that may be used include but are not limited to herbicides for example, plant growth regulators Ethephon (e.g. Bromeflor, Cerone, Chlorethephon Ethrel, Florel, Prep and Flordimex), Daminozide (Butanedioic acid mono-2,2-dimethylhydrazine,-Succinic acid 2,2-dimethylhydrazide; e.g. B-nine; Alar, Kylar, SADH, B-nine, B-995, aminozide), Atrimmec (dikegulac sodium), maleic hydrazide (1,2,-dyhydro-3,6-pyridazinedione), 2-4-D (2,4, dichlorophenoxyacetic acid), and including inhibitors of gibberellic acid synthesis, for example, but not limited to Cycocel (chlormequat chloride), A-Rest (ancymidol), triazols, for example, Bonzi (paclobutrazol), Sumagic (uniconazole), or 3-Amino-1,2,4-triazole (3-AT). These compounds may be used at known dosage ranges for plant growth modification, for example the dosage range used may be those as recommended by the manufacture of the chemical compound. These compounds may be also used at dosage ranges that are below those known for plant growth modification, for example the dosage range used may be used at 75%, 50%, 25%, 10% of that recommended by the manufacture of the chemical compound. These compounds may be used from about 0.2 ppm to about 5,000ppm, and any amount therebetween, depending upon the growth regulator selected. Furthermore, the pruning agent (chemical compound) may be applied once, or additional applications may be made as required. For example, the chemical compound may be applied one time, or the chemical compound may be applied more than one time, to result in a chemical pruning of the plant prior to, or after infiltration. If chemical pruning is used, then the chemical compound may be applied from about 20 days prior to infiltration to about 2 days after infiltration or any time in between, for example application of a chemical compound at 14 days, 7 days, or 5 days prior to infiltration may effectively be used.

As shown if FIGS. 7A, 7B, 7C, 8 and 9, pruning a plant prior to infiltration results in an increase in the expression of the protein of interest. This effect is observed when either mechanical or chemical pruning methods are used. Therefore, the present invention provides a method for synthesizing a protein of interest within a plant or a portion of a plant comprising,

i) pruning the plant or portion of the plant to produce a pruned plant or portion of the plant,

ii) introducing one or more than one nucleic acid sequence encoding a protein of interest operatively linked with a regulatory region that is active in the plant, into the pruned plant or portion of the plant in a transient manner, and

iii) maintaining the pruned plant or portion of the plant under conditions that permit the nucleic acid sequence encoding the protein of interest to be expressed in the plant or a portion of the plant.

The nucleic acid sequence encoding the protein of interest may be introduced into the plant or a portion of the plant, by any suitable method as would be known by one of skill in the art, for example which is not to be considered limiting, by vacuum infiltration, or syringe infiltration. Methods of vacuum infiltration are known in the art and may include, but are not limited to Kapila et al. (1997; which is incorporated herein by reference). Infiltration also refers to introducing the nucleic acid sequence encoding the protein of interest in to a plant or a portion of a plant using syringe infiltration (Liu and Lomonossoff, 2002; which is incorporated herein by reference).

The methods used in the present invention and those previously described (for example, Kapila et al., 1997; or Liu and Lomonossoff, 2002) culture Agrobacterium in a medium comprising acetosyringone prior to its use for transient transformation. Acetosyringone or other phenolic signal molecules are known to positively regulate the vir machinery of Agrobacterium. The increased levels of expression levels of a protein of interest described herein are observed when Agrobacterium have been cultured in the presence or absence of acetosyringone.

Post-transcriptional gene silencing (PTGS) may be involved in limiting expression of transgenes in plants, and co-expression of a suppressor of silencing from the potato virus Y (HcPro) may be used to counteract the specific degradation of transgene mRNAs (Brigneti et al., 1998). Alternate suppressors of silencing are well known in the art and may be used as described herein (Chiba et al., 2006, Virology 346:7-14; which is incorporated herein by reference), for example but not limited to, TEV-p1/HC-Pro (Tobacco etch virus-p1/HC-Pro), BYV-p21, p19 of Tomato bushy stunt virus (TBSV p19), capsid protein of Tomato crinkle virus (TCV-CP), 2b of Cucumber mosaic virus; CMV-2b), p25 of Potato virus X (PVX-p25), pll of Potato virus M (PVM-p11), p11 of Potato virus S (PVS-p11), p16 of Blueberry scorch virus, (BScV-p16), p23 of Citrus tristexa virus (CTV-p23), p24 of Grapevine leafroll-associated virus-2, (GLRaV-2 p24), p10 of Grapevine virus A, (GVA-p10), p14 of Grapevine virus B (GVB-p14), p10 of Heracleum latent virus (HLV-p10), or p16 of Garlic common latent virus (GCLV-p16). Therefore, a suppressor of silencing, for example, but not limited to, HcPro, TEV-p1/HC-Pro, BYV-p21, TBSV p19, TCV-CP, CMV-2b, PVX-p25, PVM-p11, PVS-p11, BScV-p16, CTV-p23, GLRaV-2 p24, GBV-p14, HLV-p10, GCLV-pl6or GVA-p10, may be co-expressed along with the nucleic acid sequence encoding the protein of interest to further ensure high levels of protein production within a plant.

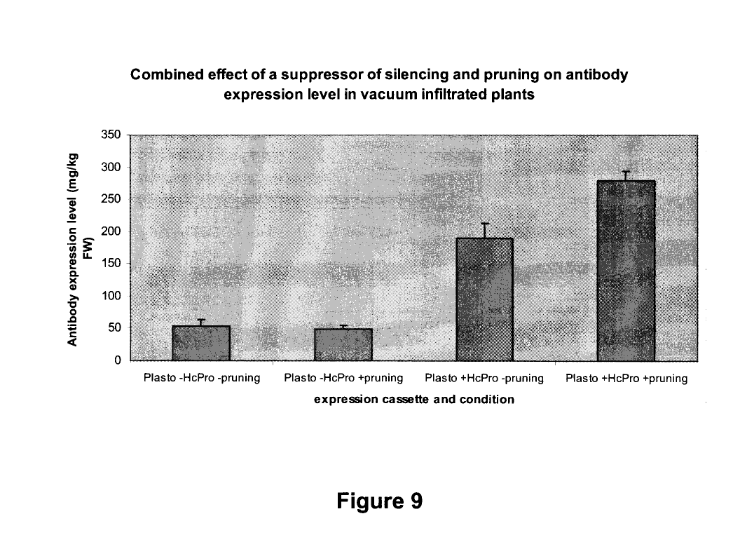

As shown in FIG. 9, the co-expression of a suppressor of silencing with the nucleic acid sequence encoding the protein of interest results in significant increase in the yield of the protein of interest. The effect is also observed if plants are pruned prior to infiltration. Therefore, the method of synthesizing a protein of interest as described herein may include the introduction of two or more than two nucleic acid sequences within the plant or portion of the plant. For example, one of the two or more than two nucleic acid sequences may encode a suppressor of silencing.

To exemplify the method of producing a protein of interest in high yield, the present invention describes a plant expression system for driving the expression of a protein of interest, for example a complex proteins such as an antibody. Expression of a complex protein within an agroinfiltrated plant, for example Nicotiana benthamiana, produced levels of protein reaching 1.5 g/kg FW (approx. 25% TSP). Average levels of 558 and 757 mg/kg/FW were attained for the secreted and ER-retained forms of the protein of interest, respectively. In the non-limiting example provided, this expression level was obtained for an antibody, at level of expression threefold higher than for an antibody produced using a multi-virus transient expression system (Giritch et al. 2006), and well above levels described for non-viral agro-infiltrated expression systems (e.g. Vaquero et al. 1999).

In the example provided herein, which is not to be considered limiting, the antibody comprises a modified glycosylation pattern with reduced fucosylated, xylosylated, or both, fucosylated and xylosylated, N-glycans. The impact of the difference between plant and typical mammalian N-glycosylation has been a major concern surrounding the concept of using plants for therapeutics production. The occurrence of plant-specific glycans may contribute to shorten the half-life of a plant-made protein in the blood stream, or that the same glycans provoke hypersensitivity reactions in patients. In this manner the protein of interest may be produced in high yield and lack glycans that may provoke hypersensitivity reactions, or be otherwise involved in allergenic reactions. However, it is to be understood that the method of transient protein production described herein may be used for any protein of interest including those that do not comprise modified glycosylation.

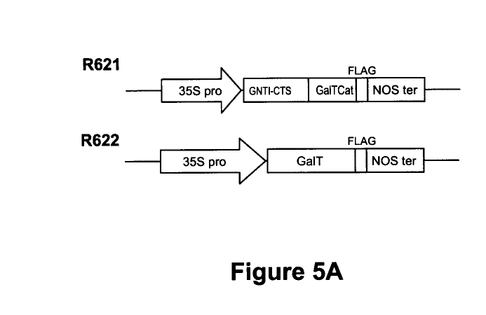

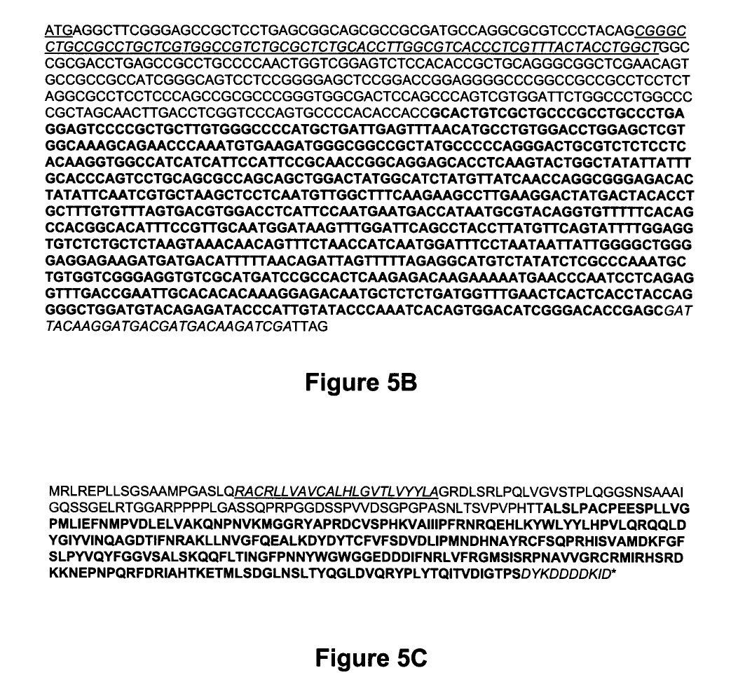

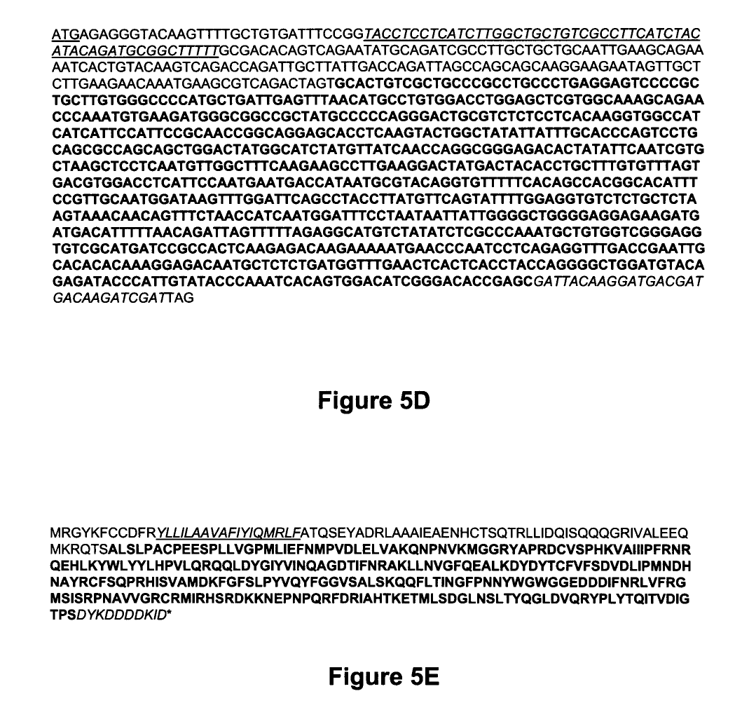

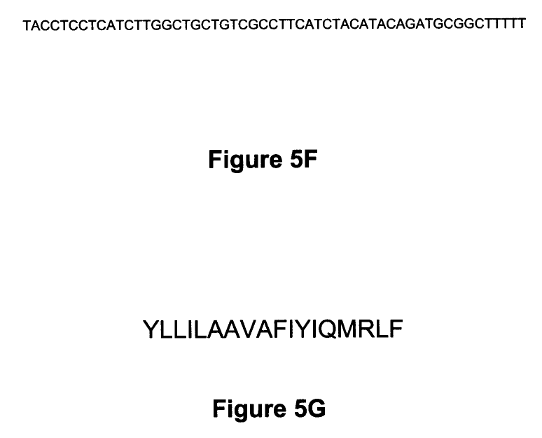

A method for the synthesis of a protein of interest within plants characterized in having a modified glycosylation pattern is described. The method involves co-expressing the protein of interest along with a nucleotide sequence expressing human beta-1.4galactosyltrasnferase (hGalT, also referred to as GaltT; SEQ ID NO:14). The hGalT may also be fused to a CTS domain (i.e. the cytoplasmic tail, transmembrane domain, stem region) of N-acetylglucosamine transferase (GNT1; SEQ ID NO:21, FIG. 5f; amino acid SEQ ID NO:22, FIG. 5g) to produce a GNT1-GalT hybrid enzyme, and the hybrid enzyme co-expressed with the protein of interest.

The use of a hybrid GNT1-GalT sequence positions the catalytic domain of the hGalT in the cis-Golgi apparatus where early stages in complex N-glycan maturation occurs. The protein of interest may also be co-expressed with a hybrid enzyme comprising a CTS domain fused to GalT, for example GNT1-GalT (R621; FIG. 5a; SEQ ID NO:18, encoded by SEQ ID NO:17). However, if a protein of interest comprising reduced levels of fucoslylation, while still comprising xylosylated and galatosylated proteins is desired, then, GalT may be co-expressed with the protein of interest.

By “modified glycosylation” of a protein of interest it is meant that the N-glycan profile of the protein of interest comprising modified glycosylation (for example, as described herein), is different from that of the N-glycan profile of the protein of interest produced in a wild-type plant. Modification of glycosylation may include an increase or a decrease in one or more than one glycan of the protein of interest. For example, the protein of interest may exhibit reduced xylosylation, reduced fucosylation, or both reduced xylosylation and reduced fucosylation. Alternatively, the N-glycan profile of the protein of interest may be modified in a manner so that the amount of galactosylation is increased, and optionally, the amount xylosylation, fucosylation, or both, are reduced.

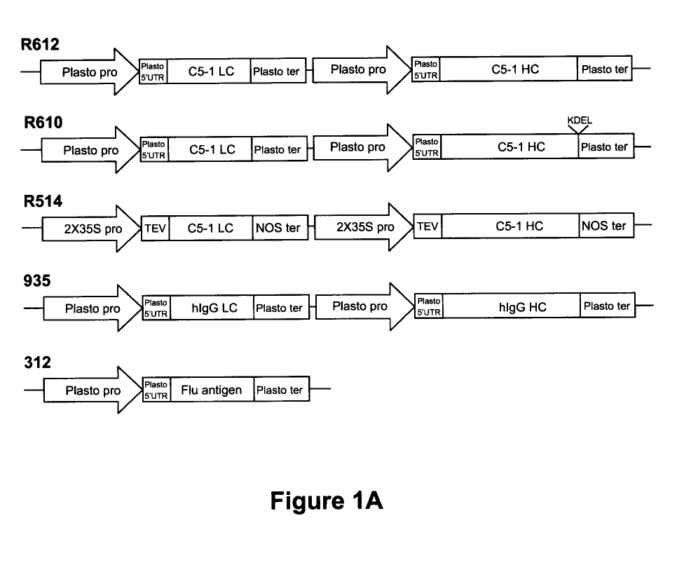

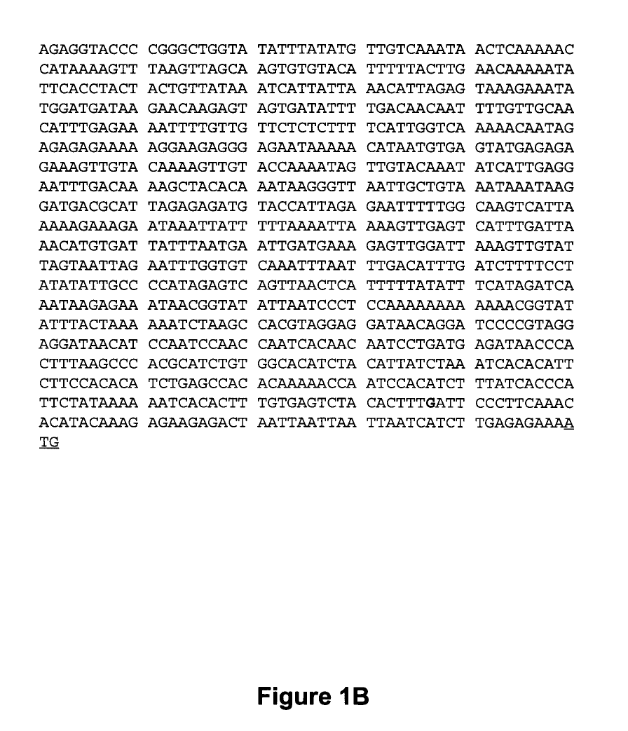

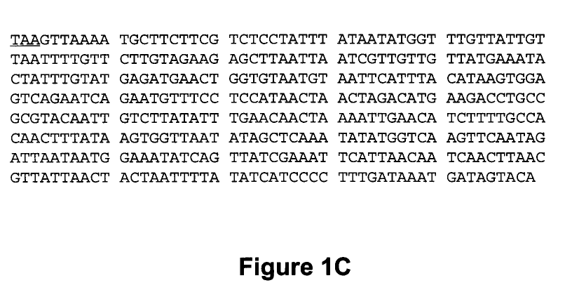

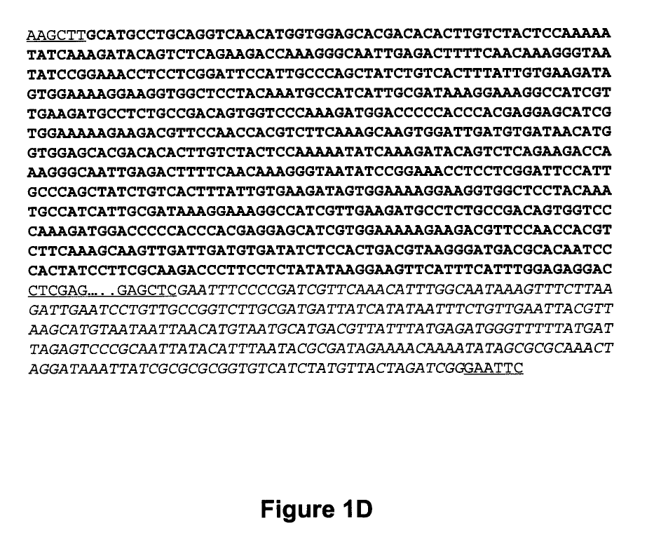

Also, when complex proteins of interest are produced the nucleotide sequence, may encode more than one peptide or domain of the complex protein. For example, in the case where the protein of interest is an antibody, the nucleotide sequence may comprise two nucleotide sequences, each encoding a portion of the antibody, for example one nucleotide sequence may encode a light chain and a second sequence encode a heavy chain of the antibody. Non-limiting examples of such constructs are provided in FIG. 1, where construct each of R612 and R610 comprise two nucleotide sequences, one encoding C5-1 LC (the light chain of C5-1) operatively linked to a regulatory region active in a plant, for example, but not limited to the plastocyanin promoter as described in U.S. Pat. No. 7,125,978 (which is incorporated herein by reference) and the second encoding the heavy chain of C5-1 (C5-1 HC) operatively linked to a regulatory region active in a plant, for example, but not limited to the plastocyanin promoter (U.S. Pat. No. 7,125,978, which is incorporated herein by reference). As shown in FIG. 1, and with reference to R610, a KDEL sequence may be fused to the c-terminal region of one of the peptides 2A or 2B, for example which is not to be considered limiting, the KDEL sequence may be fused to the heavy chain of the antibody to ensure that the antibody is retained with the ER.

Each protein encoded by the nucleotide sequence may be glycosylated.

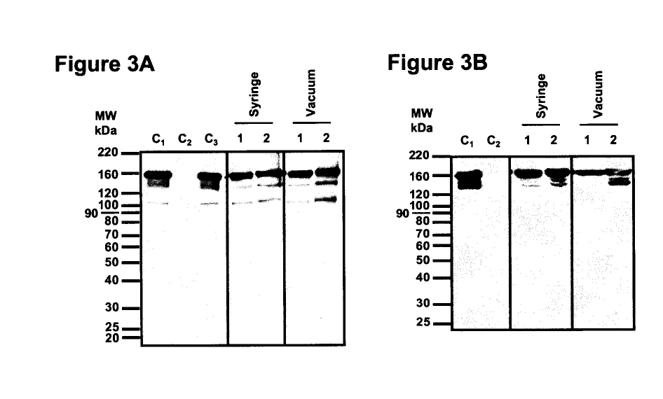

The Coomassie staining of purified products produced using transient expression shows the presence of various contaminants of low abundance. These fragments appear to be product related, and all contaminants over 70 kDa contained at least one Fab as shown by the activity blot (FIG. 3B). The identity and quantity of product related contaminants present in plant extracts being similar to those observed in mammalian cell production systems. Therefore, a purification train typically used for the purification of therapeutic antibodies (e.g. anion exchange, affinity and cation exchange) easily yields the purity required by the regulatory agencies for a protein for therapeutic use.

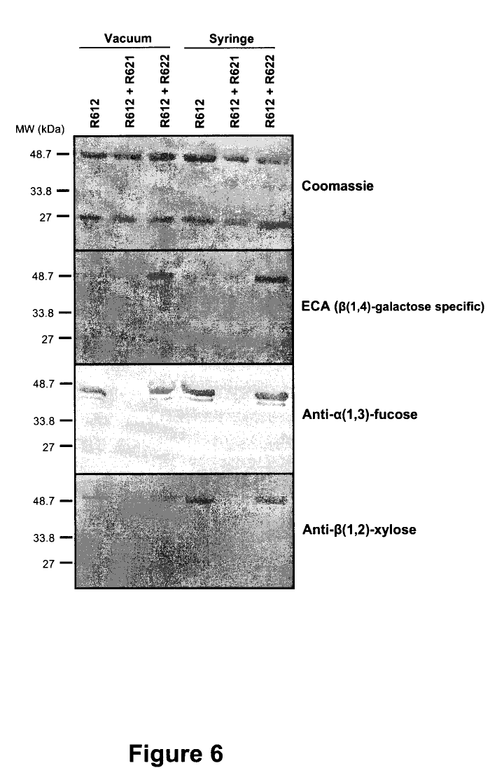

As shown in FIG. 6, by using the methods described herein, a protein of interest may be produced that exhibits a modified glycosylation profile. For example, a protein of interest with immunogenetically undetectable fucose or xylose residues has been produced when the protein of interest is co-expressed with GNT1-GalT. MALDI-TOF analysis of an epitope of a protein of interest demonstrates that a protein of interest with a modified glycosylation pattern may be obtained when the protein of interest is co-expressed with either GalT or GNT1-GalT.

The plant, portion of the plant, or plant matter, may be used as a feed, the plant or portion of the plant may be minimally processed, or the protein of interest may be extracted from the plant or portion of the plant, and if desired, the protein of interest may be isolated and purified using standard methods.

Additional modifications to the nucleotide sequence encoding the protein of interest may be made to ensure high yield. The nucleic acid sequence encoding the protein of interest may also be fused to a sequence encoding a sequence active in retaining the protein within the endoplasmic reticulum (ER), for example but not limited to, a KDEL (Lys-Asp-Glu-Leu) sequence, or other known ER retention sequences for example HDEL.

The method of protein production as described herein may involve use of a plant that may be used as a “platform” for the production of a protein of interest. For example, the platform plant typically expresses in a stable manner one or more than one protein that modifies production of the protein of interest in some manner, for example to produce the protein of interest with modified N-glycosylation. For example, the platform plant may express one or more than one first nucleotide sequence encoding GalT, GNT1-GalT, or both GalT and GNT1-GalT. To produce the protein of interest, a second nucleotide sequence encoding the protein of interest, is introduced into the platform plant using transient transformation following pruning of the plant form plant, or a portion of the platform plant, and the second nucleotide sequence is expressed so that the protein of interest produced, and in this case, comprises glycans with modified N-glycosylation. However it is to be understood that a platform plant, stably expressing other proteins, may be used to modify the protein of interest as desired. The plant or portion of the plant may be used as a feed, or the plant or portion of the plant may be minimally processed, or the protein of interest may be extracted from the plant or portion of the plant, and if desired, the protein of interest may be isolated and purified using standard methods.

The present invention provides to a method for expressing a protein of interest with a modified glycosylation using a platform plant, or portion of a platform plant, comprising a nucleotide sequence encoding GalT, GNT1-GalT, both GalT and GNT1-GalT, or a combination thereof, each operatively linked with a regulatory region that is active in the platform plant. The platform plant, or portion of the platform plant, may then be used to express a second nucleotide sequence encoding one or more than one of a protein of interest, the second nucleotide sequence operatively linked to one or more than one second regulatory region active within the platform plant. The first nucleotide sequence, the second nucleotide sequence, or both the nucleotide sequence and the second nucleotide sequence, may be codon optimized for expression within the platform plant, or portion of the platform plant. The method comprises, first pruning the platform plant or portion of the platform plant. After pruning, one or more than one second nucleic acid sequence encoding a protein of interest operatively linked with a regulatory region that is active in the plant, is introduced into the pruned plant or portion of the plant in a transient manner. The plant or portion of the plant is then maintained under conditions that permit the nucleic acid sequence encoding the protein of interest to be expressed in the plant or a portion of the plant.

The nucleotide sequences encoding the protein of interest, or the enzymes that modify glycosylation of the protein of interest, for example, GalT, GNT1-GalT, both GalT and GNT1-GalT, or a combination thereof, may be codon optimized to increase the level of expression within the plant. By codon optimization it is meant the selection of appropriate DNA nucleotides for the synthesis of oligonucleotide building blocks, and their subsequent enzymatic assembly, of a structural gene or fragment thereof in order to approach codon usage within plants. The sequence may be a synthetic sequence, optimized for codon usage within a plant using a procedure similar to that outlined by Sardana et al. (Plant Cell Reports 15:677-681; 1996). A table of codon usage from highly expressed genes of dicotyledonous plants is available from several sources including Murray et al. (Nuc Acids Res. 17:477-498; 1989). Furthermore, sequence optimization may also include the reduction of codon tandem repeats, the elimination of cryptic splice sites, the reduction of repeated sequence (including inverted repeats) and can be determined using, for example, Leto 1.0 (Entelechon, Germany).

By “operatively linked” it is meant that the particular sequences interact either directly or indirectly to carry out an intended function, such as mediation or modulation of gene expression. The interaction of operatively linked sequences may, for example, be mediated by proteins that interact with the operatively linked sequences. A transcriptional regulatory region and a sequence of interest are operably linked when the sequences are functionally connected so as to permit transcription of the sequence of interest to be mediated or modulated by the transcriptional regulatory region.

By the term “portion of a plant”, it is meant any part derived from a plant, including the entire plant, tissue obtained from the plant for example but not limited to the leaves, the leaves and stem, the roots, the aerial portion including the leaves, stem and optionally the floral portion of the plant, cells or protoplasts obtained from the plant.

By the term “plant matter”, it is meant any material derived from a plant. Plant matter may comprise an entire plant, tissue, cells, or any fraction thereof. Further, plant matter may comprise intracellular plant components, extracellular plant components, liquid or solid extracts of plants, or a combination thereof. Further, plant matter may comprise plants, plant cells, tissue, a liquid extract, or a combination thereof, from plant leaves, stems, fruit, roots or a combination thereof. Plant matter may comprise a plant or portion thereof which has not been subjected to any processing steps. However, it is also contemplated that the plant material may be subjected to minimal processing steps as defined below, or more rigorous processing, including partial or substantial protein purification using techniques commonly known within the art including, but not limited to chromatography, electrophoresis and the like.

By the term “minimal processing” it is meant plant matter, for example, a plant or portion thereof comprising a protein of interest which is partially purified to yield a plant extract, homogenate, fraction of plant homogenate or the like (i.e. minimally processed). Partial purification may comprise, but is not limited to disrupting plant cellular structures thereby creating a composition comprising soluble plant components, and insoluble plant components which may be separated for example, but not limited to, by centrifugation, filtration or a combination thereof. In this regard, proteins secreted within the extracellular space of leaf or other tissues could be readily obtained using vacuum or centrifugal extraction, or tissues could be extracted under pressure by passage through rollers or grinding or the like to squeeze or liberate the protein free from within the extracellular space. Minimal processing could also involve preparation of crude extracts of soluble proteins, since these preparations would have negligible contamination from secondary plant products. Further, minimal processing may involve aqueous extraction of soluble protein from leaves, followed by precipitation with any suitable salt. Other methods may include large scale maceration and juice extraction in order to permit the direct use of the extract.

The plant matter, in the form of plant material or tissue may be orally delivered to a subject. The plant matter may be administered as part of a dietary supplement, along with other foods, or encapsulated. The plant matter or tissue may also be concentrated to improve or increase palatability, or provided along with other materials, ingredients, or pharmaceutical excipients, as required.

It is contemplated that a plant comprising the protein of interest may be administered to a subject, for example an animal or human, in a variety of ways depending upon the need and the situation. For example, the protein of interest obtained from the plant may be extracted prior to its use in either a crude, partially purified, or purified form. If the protein is to be purified, then it may be produced in either edible or non-edible plants. Furthermore, if the protein is orally administered, the plant tissue may be harvested and directly feed to the subject, or the harvested tissue may be dried prior to feeding, or an animal may be permitted to graze on the plant with no prior harvest taking place. It is also considered within the scope of this invention for the harvested plant tissues to be provided as a food supplement within animal feed. If the plant tissue is being feed to an animal with little or not further processing it is preferred that the plant tissue being administered is edible.

As described in more detail in the Examples, GalT, GNT1-GalT, and the protein of interest were introduced into plants in a transient manner. Immunological analysis, using appropriate antibodies, demonstrated that a protein of MWᵣ 150 kDa was present in the transformed cells (FIGS. 2, 3A and 3B). Furthermore GalT or GNT1-GaIT was detectable in extracts obtained from plants expressing either construct, and altered N glycosylation of a protein of interest was observed when GNT1-GalT was expressed in the plant (FIG. 6). Therefore, recombinantly expressed GlaT, or GNT1-GalT is biologically active in planta.

An “analogue” or “derivative” includes any substitution, deletion, or addition to the nucleotide sequence encoding GalT (SEQ ID NO:14) or GNT1-GalT (SEQ ID NO:17), provided that the sequence encodes a protein that modifies the glycosylation profile of a protein of interest, for example reducing the fucosylation, xylosylation, or both, of glycans of the protein of interest, or increasing the galactosylation of the protein of interest when compared to the glycoslylation profile of the protein of interest produced in the absence of GalT (SEQ ID NO:14) or GNT1-GalT (SEQ ID NO:17). For example the protein encoded by the sequence may add a terminal galactose during N glycan maturation. Derivatives, and analogues of nucleic acid sequences typically exhibit greater than 80% similarity (or identity) with, a nucleic acid sequence.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region), when compared and aligned for maximum correspondence over a comparison window, or designated region as measured using a sequence comparison algorithms (for example Altschul et al., Nuc. Acids Res. 25:3389-3402 (1977) and Altschul et al., J. Mol. Biol. 215:403-410 (1990), and any upgrades to these algorithms), or by manual alignment and visual inspection. Sequence similarity, may be determined by use of the BLAST algorithm (GenBank: ncbi.nlm.nih.gov/cgi- bin/BLAST/), using default parameters (Program: blastn; Database: nr; Expect 10; filter: low complexity; Alignment: pairwise; Word size:11).

Analogs, or derivatives thereof, also include those nucleotide sequences that hybridize under stringent hybridization conditions (see Maniatis et al., in Molecular Cloning (A Laboratory Manual), Cold Spring Harbor Laboratory, 1982, p. 387-389, or Ausubel, et al. (eds), 1989, Current Protocols in Molecular Biology, Vol. 1, Green Publishing Associates, Inc., and John Wiley & Sons, Inc., New York, at p. 2.10.3) to any one of the GalT (SEQ ID NO:14), GNAT1-GalT (SEQ ID NO:17) sequences described herein, provided that the sequence encodes a protein that modifies the glycosylation profile of a protein of interest, for example reducing the fucosylation, xylosylation, or both, of glycans of the protein of interest, or increasing the galactosylation of the protein of interest when compared to the glycoslylation profile of the protein of interest produced in the absence of GaIT (SEQ ID NO:14) or GNT1-GalT (SEQ ID NO:17). For example the protein encoded by the sequence may add a terminal galactose during N glycan maturation. An example of one such stringent hybridization conditions may be hybridization with a suitable probe, for example but not limited to, a [gama-³²P]dATP labelled probe for 16-20 hrs at 65C in 7% SDS, 1 mM EDTA, 0.5M Na₂HPO₄, pH 7.2. Followed by washing at 65C in 5% SDS, 1 mM EDTA 40 mM Na₂HPO₄, pH 7.2 for 30 min followed by washing at 65C in 1% SDS, 1 mM EDTA 40 mM Na₂HPO₄, pH 7.2 for 30 min. Washing in this buffer may be repeated to reduce background.

By “regulatory region” “regulatory element” or “promoter” it is meant a portion of nucleic acid typically, but not always, upstream of the protein coding region of a gene, which may be comprised of either DNA or RNA, or both DNA and RNA. When a regulatory region is active, and in operative association, or operatively linked, with a gene of interest, this may result in expression of the gene of interest. A regulatory element may be capable of mediating organ specificity, or controlling developmental or temporal gene activation. A “regulatory region” includes promoter elements, core promoter elements exhibiting a basal promoter activity, elements that are inducible in response to an external stimulus, elements that mediate promoter activity such as negative regulatory elements or transcriptional enhancers. “Regulatory region”, as used herein, also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element comprises a basal promoter element, responsible for the initiation of transcription, as well as other regulatory elements (as listed above) that modify gene expression.

A constitutive regulatory region directs the expression of a gene throughout the various parts of a plant and continuously throughout plant development. Examples of known constitutive regulatory elements include promoters associated with the CaMV 35S transcript. (Odell et al., 1985, Nature, 313: 810-812), the rice actin 1 (Thang et al, 1991, Plant Cell, 3: 1155-1165), actin 2 (An et al., 1996, Plant J., 10: 107-121), or tms 2 (U.S. Pat. No. 5,428,147, which is incorporated herein by reference), and triosephosphate isomerase 1 (Xu et. al., 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646), and the tobacco translational initiation factor 4A gene (Mandel et al, 1995 Plant Mol. Biol. 29: 995-1004). The term “constitutive” as used herein does not necessarily indicate that a gene under control of the constitutive regulatory region is expressed at the same level in all cell types, but that the gene is expressed in a wide range of cell types even though variation in abundance is often observed.

A regulatory region or promoter obtained from a photosynthetic gene is also suitable for use in the present invention. For example, regulatory regions or promoters may be obtained from the gene encoding the large or small subunit of ribulose 1,5-bisphosphate carboxylase/oxygenase (rubisco; U.S. Pat. No. 4,962,028; which is incorporated herein by reference), plastocyanin, (U.S. Pat. No. 7,125,978; which is incorporated herein by reference; FIG. 1b; SEQ ID NO:19), chlorophyll a/b binding protein (CAB; Leutwiler et a; 1986; which is incorporated herein by reference), ST-LS1 (associated with the oxygen-evolving complex of photosystem II, Stockhaus et al. 1989; which is incorporated herein by reference).

The one or more than one nucleotide sequence of the present invention may be expressed in any suitable plant host. Examples of suitable hosts include, but are not limited to, Arabidopsis, alfalfa, canola, Brassica spp., maize, Nicotiana spp, including Nicotiana benthamiana, Nicotiana tabaccum, alfalfa, potato, ginseng, pea, oat, rice, soybean, wheat, barley, sunflower, cotton and the like.

The one or more chimeric genetic constructs of the present invention can further comprise a 3′ untranslated region. A 3′ untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon. One or more of the chimeric genetic constructs of the present invention can also include further enhancers, either translation or transcription enhancers, as may be required. These enhancer regions are well known to persons skilled in the art, and can include the ATG initiation codon and adjacent sequences. The initiation codon must be in phase with the reading frame of the coding sequence to ensure translation of the entire sequence.

Non-limiting examples of suitable 3′ regions are the 3′ transcribed non-translated regions containing a 3′ UTR from platsocyanin, including the transcription termination sequence (SEQ ID NO: 20), a polyadenylation signal of Agrobacterium tumor inducing (Ti) plasmid genes (as known in the art), such as the nopaline synthase (Nos gene) and plant genes such as the soybean storage protein genes and the small subunit of the ribulose-1,5-bisphosphate carboxylase (ssRUBISCO) gene.

If desired, the constructs of this invention may be further manipulated to include selectable markers. However, this may not be required. Useful selectable markers include enzymes that provide for resistance to chemicals such as an antibiotic for example, gentamycin, hygromycin, kanamycin, or herbicides such as phosphinothrycin, glyphosate, chlorosulfuron, and the like. Similarly, enzymes providing for production of a compound identifiable by colour change such as GUS (beta-glucuronidase), or luminescence, such as luciferase or GFP, may be used.

Also considered part of this invention are transgenic plants, plant cells or seeds containing the chimeric gene construct of the present invention that may be used as a platform plant suitable for transient protein expression described herein. Methods of regenerating whole plants from plant cells are also known in the art. In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques. Transgenic plants can also be generated without using tissue cultures.

Methods for stable transformation, and regeneration of these organisms are established in the art and known to one of skill in the art. The method of obtaining transformed and regenerated plants is not critical to the present invention.

By “transformation” it is meant the interspecific transfer of genetic information (nucleotide sequence) that is manifested genotypically, phenotypically, or both. The interspecific transfer of genetic information from a chimeric construct to a host may be heritable and the transfer of genetic information considered stable, or the transfer may be transient and the transfer of genetic information is not inheritable.

The present invention further includes a suitable vector comprising the chimeric construct suitable for use with either stable or transient expression systems. The genetic information may be also provided within one or more than one construct. For example, a nucleotide sequence encoding a protein of interest may be introduced in one construct, and a second nucleotide sequence encoding a protein that modifies glycosylation of the protein of interest may be introduced using a separate construct. These nucleotide sequences may then be transiently co-expressed within a plant as described herein. A construct comprising a nucleotide sequence encoding both the protein of interest and the protein that modifies glycosylation profile of the protein of interest may also be used. In this case the nucleotide sequence would comprise a first sequence comprising a first nucleic acid sequence encoding the protein of interest operatively linked to a promoter or regulatory region, and a second sequence comprising a second nucleic acid sequence encoding the protein that modifies the glycosylation profile of the protein of interest, the second sequence operatively linked to a promoter or regulatory region.

By “co-expressed” it is meant that two or more than two nucleotide sequences are expressed at about the same time within the plant, and within the same tissue of the plant. However, the nucleotide sequences need not be expressed at exactly the same time. Rather, the two or more nucleotide sequences are expressed in a manner such that the encoded products have a chance to interact. For example, the protein that modifies glycosylation of the protein of interest may be expressed either before or during the period when the protein of interest is expressed so that modification of the glycosylation of the protein of interest takes place. The two or more than two nucleotide sequences can be co-expressed using a transient expression system, where the two or more sequences are introduced within the plant at about the same time under conditions that both sequences are expressed. Alternatively, a platform plant comprising one of the nucleotide sequences, for example the sequence encoding the protein that modifies the glycosylation profile of the protein of interest, may be transformed in a stable manner, with an additional sequence encoding the protein of interest introduced into the platform plant in a transient manner. In this case, the sequence encoding the protein that modifies the glycosylation profile of the protein of interest may be expressed within a desired tissue, during a desired stage of development, or its expression may be induced using an inducible promoter, and the additional sequence encoding the protein of interest may be expressed under similar conditions and in the same tissue, to ensure that the nucleotide sequences are co-expressed.

The constructs of the present invention can be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. D T. Dennis, D H Turpin, D D Lefebrve, D B Layzell (eds), Addison Wesly, Langmans Ltd. London, pp. 561-579 (1997). Other methods include direct DNA uptake, the use of liposomes, electroporation, for example using protoplasts, micro-injection, microprojectiles or whiskers, and vacuum infiltration. See, for example, Bilang, et al. (Gene 100: 247-250 (1991), Scheid et al. (Mol. Gen. Genet. 228: 104-112, 1991), Guerche et al. (Plant Science 52: 111-116, 1987), Neuhause et al. (Theor. Appl Genet. 75: 30-36, 1987), Klein et al., Nature 327: 70-73 (1987); Howell et al. (Science 208: 1265, 1980), Horsch et al. (Science 227: 1229-1231, 1985), DeBlock et al., Plant Physiology 91: 694-701, 1989), Methods for Plant Molecular Biology (Weissbach and Weissbach, eds., Academic Press Inc., 1988), Methods in Plant Molecular Biology (Schuler and Zielinski, eds., Academic Press Inc., 1989), Liu and Lomonossoff (J Virol Meth, 105:343-348, 2002,), U.S. Pat. Nos. 4,945,050; 5,036,006; and 5,100,792, U.S. patent application Ser. Nos. 08/438,666, filed May 10, 1995, and Ser. No. 07/951,715, filed Sep. 25, 1992, (all of which are hereby incorporated by reference).

As described below, transient expression methods may be used to express the constructs of the present invention (see Liu and Lomonossoff, 2002, Journal of Virological Methods, 105:343-348; which is incorporated herein by reference). Alternatively, a vacuum-based transient expression method, as described by Kapila et al., 1997, which is incorporated herein by reference) may be used. These methods may include, for example, but are not limited to, a method of Agro-inoculation or Agro-infiltration, syringe infiltration, however, other transient methods may also be used as noted above. With Agro-inoculation, Agro-infiltration, or synringe infiltration, a mixture of Agrobacteria comprising the desired nucleic acid enter the intercellular spaces of a tissue, for example the leaves, aerial portion of the plant (including stem, leaves and flower), other portion of the plant (stem, root, flower), or the whole plant. After crossing the epidermis the Agrobacteria infect and transfer t-DNA copies into the cells. The t-DNA is episomally transcribed and the mRNA translated, leading to the production of the protein of interest in infected cells, however, the passage of t-DNA inside the nucleus is transient.

By “gene of interest”, “nucleotide sequence of interest”, or “coding region of interest” (these terms are used interchangeably), it is meant any gene, nucleotide sequence, or coding region that is to be expressed within a plant or portion of the plant. Such a nucleotide sequence of interest may include, but is not limited to, a sequence or coding region whose product is a protein of interest. Examples of a protein of interest include, for example but not limited to, an industrial enzyme for example, cellulase, xylanase, protease, peroxidase, subtilisin, a protein supplement, a nutraceutical, a value-added product, or a fragment thereof for feed, food, or both feed and food use, a pharmaceutically active protein, for example but not limited to growth factors, growth regulators, antibodies, antigens, and fragments thereof, or their derivatives useful for immunization or vaccination and the like. Additional proteins of interest may include, but are not limited to, interleukins, for example one or more than one of IL-1 to IL-24, IL-26 and IL-27, cytokines, Erythropoietin (EPO), insulin, G-CSF, GM-CSF, hPG-CSF, M-CSF or combinations thereof, interferons, for example, interferon-alpha, interferon-beta, interferon-gama, blood clotting factors, for example, Factor VIII, Factor IX, or tPA hGH, receptors, receptor agonists, antibodies, neuropolypeptides, insulin, vaccines, growth factors for example but not limited to epidermal growth factor, keratinocyte growth factor, transformation growth factor, growth regulators, antigens, autoantigens, fragments thereof, or combinations thereof.

If the nucleotide sequence of interest encodes a product that is directly or indirectly toxic to the plant, then by using the method of the present invention, such toxicity may be reduced throughout the plant by transiently expressing the gene of interest.

As described in more detail in the examples below, synthesis of a protein of interest, for example but not limited to an antibody, C5-1, with a modified N-glycosylation was produced in a plant transiently co-expressing either GalT (SEQ ID NO:14; FIG. 1b), or GNT1-GalT (SEQ ID NO:17; FIG. 1c).

An advantage of the process of transient expression as described herein, is that the number of Agrobacterium strains used for the transient expression of antibodies is minimized, which reduces the cost, simplifies the operations, and increases robustness of the system. The transient expression system proposed by Giritch et al. (2006) relies on the expression of the light and heavy chains of antibodies on two non-competing viral vectors. This system also requires the co-infiltration of 6 different Agrobacterium cultures for the expression of provector modules, a recombinase, and two viral replicases. From a commercial perspective the simultaneous preparation of six inocula represents a significant cost in equipment, and time for the validation and to scale-up operations. In addition, multiplying the number of bacterial vectors may impact the robustness of the expression system which relies on the coordinate expression of multiple transgenes.

By comparison, the system proposed here requires the co-infiltration of only two different Agrobacterium cultures. The number of Agrobacterium cultures can be reduced to a single culture by including a sequence encoding a suppressor of silencing, for example HcPro, or any other sequences to modify the protein of interest, within the same plasmid as the antibody expression cassette.

### Listing of Sequences:

| Sequence | SEQ ID NO: |
|---|---|
| XmaI-pPlas.c | 1 |
| SacI-ATG-pPlas.r | 2 |
| SacI-PlasTer.c | 3 |
| EcoRI-PlasTer.r | 4 |
| Plasto-443c | 5 |
| Plas + LC-C51.r | 6 |
| LC-C51.c | 7 |
| LC-C51XhoSac.r | 8 |
| Plas + HC-C51.r | 9 |
| HC-C51.c | 10 |
| HC-C51XhoSac.r | 11 |
| HC- | 12 |
| C51KDEL(SacI).r | |
| tryptic | 13 |
| glycopeptide | |
| GalT (nucleotide) | 14 |
| GalT (amino acid) | 15 |
| TEV + HC-C51.r | 16 |
| GNT1-GalT | 17 |
| (nucleotide) | |
| GNT-GalT (amino acid) | 18 |
| Plastocyanin promoter | 19 |
| and 5′UTR | |
| Plastocyanin 3′ UTR and | 20 |
| terminator | |
| CTS domain of | 21 |
| GNT1(nucleotide) | |
| CTS domain of GNT1 | 22 |
| (amino acid) | |
| XhoTEV.c | 23 |
| TEV + LC-C5-1.r | 24 |
| LC-C5-1.c | 25 |
| LC-C5-1SphSac.r | 26 |
| FgalT | 27 |
| RgalTFlagStu | 28 |
| FGNT | 29 |
| RGNTSpe | 30 |
| FgalTSpe | 31 |
| HC-C51SphSac.r | 32 |
| 2X35 promoter | 33 |
| NOS terminator | 34 |

### Examples

### Example 1

### Assembly of Expression Cassettes R610, R612, R514 (FIG. 1), R621 and R622 (FIG. 5)

All manipulations were done using general molecular biology protocols from Sambrook and Russel (2001).

Oligonucleotide primers used are presented below:

SEQ ID NO: 1

XmaI-pPlas.c:

SEQ ID NO: 1

5′-AGTTCCCCGGGCTGGTATATTTATATGTTGTC-3′

SEQ ID NO: 2

SacI-ATG-pPlas.r:

SEQ ID NO: 2

5′-AATAGAGCTCCATTTTCTCTCAAGATGATTAATTAATTAATTAGTC-
3′

SEQ ID NO: 3

SacI-PlasTer.c:

SEQ ID NO: 3

5′-AATAGAGCTCGTTAAAATGCTTCTTCGTCTCCTATTTATAATATGG-
3′

SEQ ID NO: 4

EcoRI-PlasTer.r:

SEQ ID NO: 4

5′-TTACGAATTCTCCTTCCTAATTGGTGTACTATCATTTATCAAAGGGG
A-3′

SEQ ID NO: 5

Plasto-443c:

SEQ ID NO: 5

5′-GTATTAGTAATTAGAATTTGGTGTC-3′

SEQ ID NO: 6

Plas + LC-C51.r:

SEQ ID NO: 6

5′-ATCTGAGGTGTGAAAACCATTTTCTCTCAAGATG-3′

SEQ ID NO: 7

LC-C51.c:

SEQ ID NO: 7

5′-ATGGTTTTCACACCTCAGATACTTGG-3′

SEQ ID NO: 8

LC-C51XhoSac.r:

SEQ ID NO: 8

5′-ATATGAGCTCCTCGAGCTAACACTCATTCCTGTTGAAGC-3′

SEQ ID NO: 9

Plas + HC-C51.r:

SEQ ID NO: 9

5′-CAAGGTCCACACCCAAGCCATTTTCTCTCAAGATG-3′

SEQ ID NO: 10

HC-C51.c:

SEQ ID NO: 10

5′-ATGGCTTGGGTGTGGACCTTGC-3′

SEQ ID NO: 11

HC-C51XhoSac.r:

SEQ ID NO: 11

5′-ATAAGAGCTCCTCGAGTCATTTACCAGGAGAGTGGG-3′

SEQ ID NO: 12

HC-C51KDEL(SacI).r:

SEQ ID NO: 12

5′-ATAAGAGCTCTCAAAGTTCATCCTTTTTACCAGGAGAGTGGG-3′

SEQ ID NO: 23

XhoTEV.c:

SEQ ID NO: 23

5′-TTTGGAGAGGACCTCGAGAAATAACAAATCTCAACAC-3′

SEQ ID NO: 24

TEV + LC-C5-1.r:

SEQ ID NO: 24

5′-ATCTGAGGTGTGAAACCATTGCTATCGTTCGTAAATGGTG-3′

SEQ ID NO: 25

LC-C5-1.c:

SEQ ID NO: 125

5′-ATGGTTTTCACACCTCAGATACTTGG-3′

SEQ ID NO: 26

LC-C5-1SphSac.r:

SEQ ID NO: 26

5′-ATATGAGCTGCGATGCCTAACACTCATTCCTGTTGAAGC-3′

The first cloning step consisted in assembling a receptor plasmid containing upstream and downstream regulatory elements of the alfalfa plastocyanin gene. The plastocyanin promoter (U.S. Pat. No. 7,125,978, which is incorporated herein by reference) and 5′UTR sequences were amplified from alfalfa genomic DNA using oligonucleotide primers XmaI-pPlas.c (SEQ ID NO:1) and SacI-ATG-pPlass (SEQ ID NO:2). The resulting amplification product was digested with XmaI and SacI and ligated into pCAMBIA2300, previously digested with the same enzymes, to create pCAMBIA-PromoPlasto. Similarly, the 3′UTR sequences and terminator, of the plastocyanin gene (FIG. 1c; nucleotides 1-399 of SEQ ID NO:20) was amplified from alfalfa genomic DNA using the following primers: SacI-PlasTer.c (SEQ ID NO:3) and EcoRI-PlasTer.r, (SEQ ID NO:4) and the product was digested with SacI and EcoRI before being inserted into the same sites of pCAMBIA-PromoPlasto to create pCAMBIAPlasto.

Plasmids R 610 and R 612 were prepared so as to contain a C5-1 light- and a C5-1 heavy-chain coding sequences under the plastocyanin promoter of alfalfa as tandem constructs; R 610 was designed to allow retention in the ER of the assembled IgG and comprised KDEL sequence fused to the end of the heavy chain of C5-1, and R 612 was designed to allow secretion.

The assembly of C5-1 expression cassettes was performed using a PCR-mediated ligation method described by Darveau et al. (1995). To assemble the light chain coding sequences downstream of the plastocyanin promoter, a first step consisted in amplifying the first 443 base pairs (bp) of the alfalfa plastocyanin promoter (nucleotides 556-999 of FIG. 1b or SEQ ID NO:19) described by D'Aoust et al. (U.S. Pat. No. 7,125,978, which is incorporated herein by reference) downstream of the initial ATG by PCR using pCAMBIAPlasto as template and the following primers:

Plasto−443c (SEQ ID NO:5) and Plas+LC-C51.r (SEQ ID NO:6; overlap is underlined, above).

In parallel, the light chain coding sequence was PCR-amplified from plasmid pGA643-kappa (Khoudi et al., 1999) with primers the following primers:

LC-C51.c (SEQ ID NO: 7) and LC-C51XhoSac.r. (SEQ ID NO:8; overlap is underlined).

The two amplification products obtained were mixed together and used as template in a third PCR reaction using primers Plasto−443c (SEQ ID NO:5) and LC-C51XhoSacs (SEQ ID NO:8). The overlap between primers Plas+LC-C51.r (SEQ ID NO:6) and LC-C51.c (SEQ ID NO:7) used in the first reactions lead to the assembly of the amplification products during the third reaction. The assembled product resulting from the third PCR reaction was digested with DraIII and SacI and ligated in pCAMBIAPlasto digested with DraIII and SacI to generate plasmid R540.

The heavy chain coding sequence was fused to plastocyanin upstream regulatory element by amplifying the 443 by upstream of the initial ATG of plastocyanin, nucleotides 556-999 of (FIG. 1b; SEQ ID NO:19), by PCR using pCAMBIAPlasto as template with the following primers:

Plasto−443c (SEQ ID NO:5) and Plas+HC-C51.r (SEQ ID NO:9; overlap underlined above).

The product of these reactions were mixed and assembled in a third PCR reaction using primers Plasto−443c (SEQ ID NO:5) and HC-C51XhoSacs (SEQ ID NO:11). The resulting fragment was digested with DraIII and SacI and ligated in pCAMBIAPlasto between the DraIII and SacI sites. The resulting plasmid was named R541.

A KDEL tag was added in C-terminal of the heavy chain coding sequence by PCR-amplification with primers Plasto−443c (SEQ ID NO:5) and HC-C51KDEL (SacI).r (SEQ ID NO:12) using plasmid R541 as a template. The resulting fragment was digested with DraIII and SacI cloned into the same sites of pCAMBIAPlasto, creating plasmid R550.

Assembly of light- and heavy chain expression cassettes on the same binary plasmid was performed as follows: R541 and R550 were digested with EcoRI, blunted, digested with HindIII and ligated into the HindIII and SmaI sites of R540 to create R610 (with KDEL) and R612 (without KDEL; see FIG. 1).

R514 (FIG. 5a)

Additional oligonucleotide primers used are presented below:

SEQ ID NO: 16

Tev + HC-051.2:

SEQ ID NO: 16

5′-CAAGGTCCACACCCAAGCCATTGCTATCGTTCGTAAATGGTG-3′

SEQ ID NO: 32

HC-C51SphSac.r

SEQ ID NO: 32

5′-ATAAGAGCTCGCATGCTCATTTACCAGGAGAGTGGG-3′

Full-length C5-1 light and heavy chains gene (LC and HC) were provided by Héma-Québec and were cloned in-frame into plant binary expression vector using the polymerase chain reaction (PCR)-mediated method described by Darveau (1995). The tobacco etch virus (TEV) enhancer was first amplified by RT-PCR on TEV genomic RNA (Acc. No. NC001555) with primers XhoTEV.c (SEQ ID NO:23) and TEV+LC-C51 (SEQ ID NO:24). In parallel C5-1 light chain coding sequence was amplified by PCR from plasmid pGA643 (Khoudi et al., 1999) with primers LC-C51.c (SEQ ID NO:25) and LC-C51SphSac.r (SEQ ID NO:26) for LC. The TEV and light chain amplification fragments were then mixed together and assembled by another round of PCR using XhoTEV.c (SEQ ID NO:23) and LC-C51SphSac.r (SEQ ID NO:26) as primers. The resulting TEV/C5-1LC fragment was then purified and cloned as XhoI-SacI digest in an intermediary vector between the 2X355 promoter and the NOS terminator. FIG. 1d presents the sequence of the 2X35S promoter (in bold; (SEQ ID NO:33)), and the NOS terminator (in italics; (SEQ ID NO:34) used and the position of the restriction sites (underlined). This expression cassette was then transferred to the pCAMBIA2300 binary plasmid as a HindIII-EcoRI fragment to create plasmid R512.

To create pR513, the TEV enhancer was amplified by RT-PCR on TEV genomic RNA (Acc. No. NC001555) with primers XhoTEV.c (SEQ ID NO:23) and TEV+HC-C51.r (SEQ ID NO:16). In parallel, the coding sequence for the heavy chain of the antibody was amplified by PCR with primers HC-C51.c (SEQ ID NO:10) and HC-C51SphSac.r (SEQ ID NO:32). The resulting TEV and heavy chain fragments mixed together and assembled by PCR with primers XhoTEV.c (SEQ ID NO:23) and HC-C51SphSac.r (SEQ ID NO:32). The resulting TEV/C5-1HC fragment was purified, digested with XhoI and SacI, and cloned into the same sites of intermediary vector between the 2X35S promoter and the NOS terminator. FIG. 1d presents the sequence of the 2X35S (SEQ ID NO:33) promoter used, the NOS terminator (SEQ ID NO:34) used and the position of the restriction sites. The resulting plasmid containing the 2X35S/TEV/C5-1HC/NOS fragment was digested with EcoRI, blunt ended with the Klenow fragment polymerase, and further digested with HindIII. This HindIII-EcoRI (blunt) fragment was then ligated into a HindIII-SmaI digest of R512 to create plasmid R514.

R621 and R622 (FIG. 5a)—Oligonucleotide Primers Used are Presented Below:

SEQ ID NO: 27

FgalT

SEQ ID NO: 27

5′-GACTCTAGAGCGGGAAGATGAGGCTTCGGGAGCCGCTC-3′

SEQ ID NO: 28

RgalTFlagStu

SEQ ID NO: 28

5′- AAGGCCTACG CTACTTGTCAT CGTCATCTTT GTAGTCGCAC
GGTGTCCCG AAGTCCAC -3′

SEQ ID NO: 29

FGNT

SEQ ID NO: 29

5′-ATCGAAATCGCACGATGAGAGGGAACAAGTTTTGC-3′

SEQ ID NO: 30

RGNTSpe

SEQ ID NO: 30

5′-CGGGATCCACTAGTCTGACGCTTCATTTGTTCTTC-3′

SEQ ID NO: 31

FgalTSpe

SEQ ID NO: 31

5′-GGACTAGTGCACTGTCGCTGCCCGCCTGC-3′

Plasmids for GalT and GNTIGalT expression were assembled from pBLTI121 (Pagny et al., 2003). The human β(1,4)-galactosyltransferase (hGalT) gene (UDP galactose: β-N-acetylglucosaminide: β(1,4)-galactosyltransferase; EC 2.4.1.22) was isolated from pUC19-hGalT (Watzele et al.,1991) with EcoRI digestion. After klenow treatment, the 1.2-kb hGalT fragment was cloned into pBLTI221 at Sma I sites, resulting in plasmid pBLTI221hGalT. A flag tag was then fused to the C-terminal end of the coding region by PCR using primers FGalT (SEQ ID NO: 27) and RGalTFlagStu (SEQ ID NO: 28) for amplification. R622 was then produced by cloning this XbaI-StuI fragment into the binary vector pBLTI121. The first 77 a.a. from N-acetylglucosaminyltransferase I (GNTI) corresponding to the transmembrane domain were amplified by PCR using the N. tabacum cDNA encoding N-GNTI as template (Strasser et al, 1999) and FGNT (SEQ ID NO: 29) and RGNTSpe (SEQ ID NO: 30) as primers. The amplification product was first cloned into pGEM-T vector, and the resulting plasmid was digested with ApaI and BamHI, and ligated into pBLTI221, producing a plasmid named pBLTI221-GNTI. The catalytic domain of hGalT was obtained by PCR amplification on pBLTI221hGalT using primers FGalTSpe (SEQ ID NO: 31) and RgalTFlagStu (SEQ ID NO: 28), creating SpeI and Stul sites in 5′ and 3′ end, respectively. The SpeI/Stul hGalT fragment was then cloned into pBLTI221-GNTI using the same (SpeI and StuI) sites, creating pBLTI221-GNTIGalT. Finally, pBLTI221-GNTIGalT was digested with XbaI and StuI isolating the GNTIGalT coding sequence (FIG. 5d; SEQ ID NO: 17), and R621 was produced by cloning this fragment into the binary vector pBLTI121.

All clones were sequenced to confirm the integrity of the constructs. The plasmids were used to transform Agrobacteium tumefaciens (AGL1; ATCC, Manassas, Va. 20108, USA) by electroporation (Hofgen and Willmitzer, 1988) using a Gene Pulser II apparatus (Biorad, Hercules, Calif., USA) as for E. coli transformation (W. S. Dower, Electroporation of bacteria, In “Genetic Engineering”, Volume 12, Plenum Press, New York, 1990, J. K. Setlow eds.). The integrity of all A. tumefaciens strains were confirmed by restriction mapping.

An HcPro construct was prepared as described in Hamilton et al. (2002).

### Example 2

### Preparation of Plant Biomass, Inoculum, Agroinfiltration, and Harvesting

Nicotiana benthamiana plants were grown from seeds in flats filled with a commercial peat moss substrate. The plants were allowed to grow in the greenhouse under a 16/8 photoperiod and a temperature regime of 25° C. day/20° C. night. Three weeks after seeding, individual plantlets were picked out, transplanted in pots and left to grow in the greenhouse for three additional weeks under the same environmental conditions. Prior to transformation, apical and axillary buds were removed at various times as indicated below, either by pinching the buds from the plant, or by chemically treating the plant

Agrobacteria strains R612, R610, R621, R622 or 35SHcPro were grown in a YEB medium supplemented with 10 mM 2-[N-morpholino]ethanesulfonic acid (MES), 20 μM acetosyringone, 50 μg/ml kanamycin and 25 μg/ml of carbenicillin pH5.6 until they reached an OD₆₀₀ between 0.6 and 1.6. Agrobacterium suspensions were centrifuged before use and resuspended in infiltration medium (10 mM MgCl2 and 10 mM MES pH5.6).

Syringe-infiltration was performed as described by Liu and Lomonossoff (2002, Journal of Virological Methods, 105:343-348).

For vacuum-infiltration, A. tumefaciens suspensions were centrifuged, resuspended in the infiltration medium and stored overnight at 4° C. On the day of infiltration, culture batches were diluted in 2.5 culture volumes and allowed to warm before use. Whole plants of Nicotiana benthamiana were placed upside down in the bacterial suspension in an air-tight stainless steel tank under a vacuum of 20-40 Torr for 2-min. Following syringe or vacuum infiltration, plants were returned to the greenhouse for a 4-5 day incubation period until harvest.

### Leaf Sampling and Total Protein Extraction

Following incubation, the aerial part of plants was harvested, frozen at −80° C., crushed into pieces and separated into 1.5 or 7.5 g sub-samples. Total soluble proteins were extracted by homogenizing (Polytron) each sub-sample of frozen-crushed plant material in 3 volumes of cold 50 mM Tris pH 7.4, 0.15 M NaCl, 0.1% Triton X-100, 1 mM phenylmethanesulfonyl fluoride and 10 μM chymostatin. After homogenization, the slurries were centrifuged at 20,000 g for 20 min at 4° C. and these clarified crude extracts (supernatant) kept for analyses. The total protein content of clarified crude extracts was determined by the Bradford assay (Bio-Rad, Hercules, Calif.) using bovine serum albumin as the reference standard.

### Example 3

### Protein Analysis, Immunoblotting and ELISA

C5-1 is an anti-human murine IgG therefore detection and quantification can be performed through either its characteristic affinity to human IgGs (activity blots) or by its immunoreactivity to anti-mouse IgGs.

Proteins from total crude extracts or purified antibody were separated by SDS-PAGE and either stained with Coomassie Blue R-250 or G-250 or electrotransferred onto polyvinylene difluoride membranes (Roche Diagnostics Corporation, Indianapolis, Ind.) for immunodetection. Prior to immunoblotting, the membranes were blocked with 5% skim milk and 0.1% Tween-20 in Tris-buffered saline (TBS-T) for 16-18 h at 4° C.

Immunoblotting was performed by incubation with the following antibodies: a peroxidase-conjugated goat anti-mouse IgG (H+L) antibody (Jackson ImmunoResearch, West Grove, Pa., Cat#115-035-146) (0.04 μg/ml in 2% skim milk in TBS-T), a peroxidase-conjugated human IgG antibody (Gamunex® Bayer Corp., Elkhart, Ind.) (0.2 μg/ml in 2% skim milk in TBS-T) or a polyclonal goat anti-mouse IgG antibody (heavy chain specific) (Sigma-Aldrich, St-Louis, Mo.) (0.25 μg/ml in 2% skim milk in TBS-T). A peroxidase-conjugated donkey anti-goat IgG antibody (Jackson ImmunoResearch) (0.04 μg/ml in 2% skim milk in TBS-T) was used as a secondary antibody for membranes treated with the heavy chain-specific antibody. Immunoreactive complexes were detected by chemiluminescence using luminol as the substrate (Roche Diagnostics Corporation). Horseradish peroxidase-enzyme conjugation of human IgG antibody was carried out by using the EZ-Link Plus® Activated Peroxidase conjugation kit (Pierce, Rockford, Ill.).

### ELISA Quantitative Assay

Multiwell plates (Immulon 2HB, ThermoLab System, Franklin, Mass.) were coated with 2.5 μg/ml of goat anti-mouse antibody specific to IgG1 heavy chain (Sigma M8770) in 50 mM carbonate buffer (pH 9.0) at 4° C. for 16-18h. Multiwell plates were then blocked through a 1 h incubation in 1% casein in phosphate-buffered saline (PBS) (Pierce Biotechnology, Rockford, Ill.) at 37C. A standard curve was generated with dilutions of a purified mouse IgG1 control (Sigma M9269). When performing the immunoassays, all dilutions (control and samples) were performed in a plant extract obtained from plant tissue infiltrated and incubated with a mock inoculum so that any matrix effect be eliminated. Plates were incubated with protein samples and standard curve dilutions for 1 h at 37° C. After three washes with 0.1% Tween-20 in PBS (PBS-T), the plates were incubated with a peroxidase-conjugated goat anti-mouse IgG (H+L) antibody (0.04 μg/ml in blocking solution) (Jackson ImmunoResearch 115-035-146) for 1 h at 37° C. The washes with PBS-T were repeated and the plates were incubated with a 3,3′,5,5′-Tetramethylbenzidine (TMB) Sure Blue peroxidase substrate (KPL, Gaithersburg, Md.). The reaction was stopped by adding 1N HCl and the absorbance was read at 450 nm. Each sample was assayed in triplicate and the concentrations were interpolated in the linear portion of the standard curve.

### Example 4

### IgG Purification

Purification of C5-1 from leaf material involved taking frozen leaves of N. benthamiana (100-150 g), adding 20 mM sodium phosphate, 150 mM NaCl and 2 mM sodium meta-bisulfite at pH 5.8-6.0 and blending using a commercial blender for 2-3 min at room temperature. Insoluble fibres were removed by a coarse filtration on Miracloth™ (Calbiochem, San Diego, Calif.) and 10 mM phenylmethanesulphonyl fluoride (PMSF) was added to the filtrate. The extract was adjusted to pH 4.8±0.1 with 1 M HCl and clarified by centrifugation at 18 000 g for 15 min at 2-8° C. The clarified supernatant was adjusted to pH 8.0±0.1 with 2 M TRIS, clarified again by centrifugation at 18 000 g for 15 min at 2-8° C., and filtered on sequential 0.8 and 0.2 μm membranes (Pall Corporation, Canada). The filtered material was concentrated by tangential flow filtration using a 100 kDa molecular weight cut-off ultrafiltration membrane of 0.2 ft² of effective area (GE Healthcare Biosciences, Canada) to reduce the volume of the clarified material by 5 to 10-fold. The concentrated sample was then applied to a 5mm×5 cm column (1 mL column volume) of recombinant protein G-Sepharose Fast Flow (Sigma-Aldrich, St-Louis, Mo., Cat. #P4691). The column was washed with 5 column volumes of 20 mM TRIS-HCl, 150 mM NaCl pH 7.5. The antibody was eluted with 100 mM Glycine pH 2.9-3.0, and immediately brought to neutral pH by collection into tubes containing calculated volumes of 1 M TRIS-HCl pH 7.5. The pooled fractions of eluted antibody were centrifuged at 21 000 g for 15 min at 2-8° C. and stored at −80° C. until analysis. After purification, the affinity column was cleaned and stored according to manufacturer's instructions. The same chromatographic media could be reused for several purifications without significant modification of purification performances (up to 10 cycles tested).

### Example 5

### N-Glycosylation Analysis

Samples comprising C5-1 (50 μg) were run on 15% SDS/PAGE. Heavy and light chains were revealed with Coomassie blue and the protein band corresponding to the heavy chain was excised and cut into small fragments. Fragments were washed 3 times with 600 μL of a solution of 0.1M NH4HCO3/CH3CN (1/1) for 15 minutes each time and dried.

Reduction of disulfide bridges occurred by incubation of the gel fragments in 600 μL of a solution of 0.1M DTT in 0.1M NH4HCO3, at 56° C. for 45 minutes. Alkylation was carried out by adding 600 μL of a solution of iodoacetamide 55 mM in 0.1M NH4HCO3, at room temperature for 30 minutes. Supernatants were discarded and polyacrylamide fragments were washed once again in NH4HCO3 0.1M/CH3CN (1/1).

Proteins were then digested with 7.5 μg of trypsin (Promega) in 600 μL of 0.05M NH4HCO3, at 37° C. for 16 h. Two hundred μL of CH3CN were added and the supernatant was collected. Gel fragments were then washed with with 200 μL of 0.1M NH4HCO3, then with 200 μL CH3CN again and finally with 200 μL formic acid 5%. All supernatants were pooled and lyophilised.

Peptide separation by HPLC was carried out on a C18 reverse-phase column (4.5×250 mm) with a linear gradient of CH3CN in TFA 0.1%. Fractions were collected and lyophilised and analysed by MALDI-TOF-MS on a Voyager DE-Pro MALDI-TOF instrument (Applied Biosystems, USA) equipped with a 337-nm nitrogen laser. Mass spectra were performed in the reflector delayed extraction mode using alpha-cyano-4-hydroxycinnamic acid (Sigma-Aldrich) as matrix.

### Example 6

### Quantification of Transient IgG Expression in Agroinfiltrated Nicotiana Benthamiana Leaves

To test the whether the strong plastocyanin-based expression cassettes could drive high accumulation of a fully assembled IgG, the coding sequences of the light and heavy chain of C5-1, a murine anti-human IgG (Khoudi et el 1997) were assembled in tandem constructs downstream of the plastocyanin promoter and 5′ untranslated sequences, and flanked with the plastocyanin 3′ untranslated and transcription termination sequences on the same T-DNA segment of a pCambia binary plasmid as described in Example 1 and presented in FIG. 1.

In both the R612 and R610 expression cassettes (see Example 1), the light and heavy chain coding sequences contained the native signal peptide from C5-1 (Khoudi et al. 1999), but in R610 the coding sequence of a KDEL peptide was added at the C-terminal of the heavy chain to restrain movement of the assembled IgG to the Golgi apparatus.

Following the cloning steps and the transfer of plasmids in Agrobacterium tumefaciens (AGL1), every leaf of three Nicotiana benthamiana plants were syringe-infiltrated with Agrobacterium strains transformed with plasmids R612 R610, or R514 (FIG. 1), and incubated in greenhouse conditions for 6 days before analysis as described in Example 2. Following the incubation period, the leaves of each plant (approximately 20 g of biomass) were frozen, ground, and the frozen powder was mixed to produce an homogenous sample from which 2 sub-samples of 1.5 grams were taken for extraction (from each plant; see Example 3). The content in C5-1 was quantified in total protein extracts from each sample by an enzyme-linked immunosorbent assay (ELISA) using a polyclonal goat anti-mouse IgG1 heavy chain for capture and a peroxidase-conjugated goat anti-mouse IgG (H+L) for detection (see Example 3).

As shown in FIG. 2A, infiltration of R610, or R612 (both comprising the plastocyanin promoter) lead to greater levels of protein accumulation when compared to R514 (comprising 2X35s promoter) in the absence of a suppressor of silencing (HcPro). In the presence of HcPro, greatly increased levels of expression for both R610 and R612 were observed. As shown in FIG. 2B, agroinfiltration of R612 led to the accumulation of 106 mg of antibody per kg of fresh weight, while the ER-retained form of the antibody (R610) reached 211 mg/kg FW in the same conditions.

Because post-transcriptional gene silencing (PTGS) has been shown to limit the expression of transgenes in agroinfiltrated Nicotiana benthamiana plants and that co-expression of a suppressor of silencing from the potato virus Y (HcPro) counteracted the specific degradation of transgene mRNAs (Brigneti et al., 1998), co-infiltration of an HcPro construct (Hamilton et al., 2002) was tested for its efficiency at increasing expression of C5-1. The co-expression of R612 and R610 with HcPro increased antibody accumulation levels by 5.3-fold and 3.6-fold, respectively, compared to these observed in the absence of HcPro. In the presence of HcPro, plastocyanin-controlled C5-1 expression reached average values of 558 mg/kg FW with R612, and 757 mg/kg FW with R610 (FIG. 2A). Maximum C5-1 expression levels exceeded 1.5 g/kg FW (25% of total soluble proteins) in some extracts form both R612- and R610-infiltrated leaves.

In order to assess the scalability of an agroinfiltration expression system, the accumulation of C5-1 was quantified following a vacuum infiltration procedure adapted from Kapila et al. (1997). In this series of experiments, the aerial part of whole plants were vacuum-infiltrated with R612+HcPro or R610+HcPro and returned to the greenhouse for 6 days before harvest. In an effort to provide data which are representative of a large-scale production system, batches of approximately 250 g lots of leaves/petioles from several plants were frozen, ground into an homogeneous sample, and 3 sub-samples of 7.5 grams of material per batch were collected for analysis. As shown by ELISA quantification, average C5-1 accumulation levels reached 238 and 328 mg/kg FW for R612 and R610 infiltrations respectively (FIG. 2B).

### Effect of Pruning

Aprical and axillary buds of three Nicotiana benthamiana plants were either mechanically removed from plants by pinching 1, 2 or 3 days, or chemically pruned using Ethrel, B-nine (500 ppm), or A-rest (4 ppm), prior to vacuum infiltrating the leaves, with Agrobacterium strains transformed with appropriate plasmids.

Plants were then infiltrated with influenza antigen (construct 312, FIG. 1), human IgG (construct 935, FIG. 1) and incubated in greenhouse conditions for 6 days before analysis as described in Example 2. Control plants were not pruned. Following the incubation period, the leaves of each plant (approximately 20 g of biomass) were frozen, ground, and the frozen powder was mixed to produce an homogenous sample from which 2 sub-samples of 1.5 grams were taken for extraction (from each plant; see Example 3). The content in C5-1 was quantified in total protein extracts from each sample by an enzyme-linked immunosorbent assay (ELISA) using a polyclonal goat anti-mouse IgG1 heavy chain for capture and a peroxidase-conjugated goat anti-mouse IgG (H+L) for detection (see Example 3).

As shown in FIG. 7A, mechanical pruning to remove apical and axillary buds 12 hours before agroinfiltration of 312 (influenza antigen) led to an increase in the accumulation of antigen (150%) compared to the control treatment (no pruning). An increase in expression level up to 200%, was also observed using chemically pruned plants treated with several growth regulators (Ethrel, 500 ppm; B-nine, 2500 ppm; or A-rest, 4.0 ppm) known to inhibit apical dominance followed by agroinfiltration of 512. Similar results have also been observed using these and other growth regulator compounds when used at manufactures recommended rates of application 7 days prior to infiltration.

Mechanical pruning also resulted in an increase in the level of expression of immunoglobulin 935 (hIgG, FIG. 1) when plants were mechanically pruned 12 hours agroinfiltration, either by vacuum infiltration (FIG. 7B), or syringe infiltration (FIG. 7C).

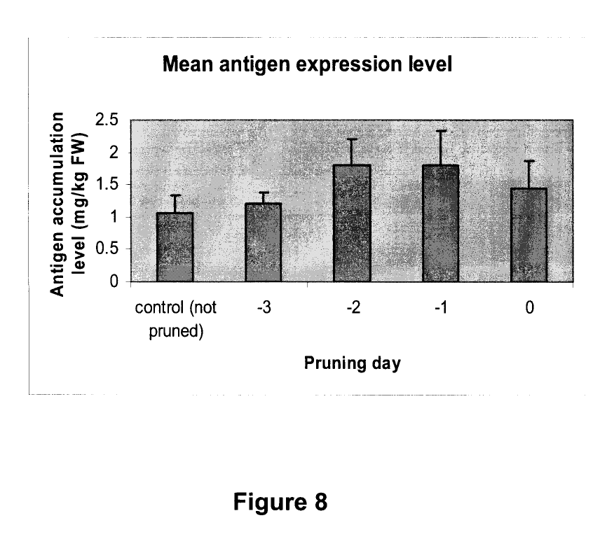

Pruning plants from one to three days before agroinfiltration resulted in an additional increase in protein (influenza antigen; 312 FIG. 1) accumulation as shown in FIG. 8 (mechanically pruned plants). A pronounced increase in expression is observed when plants are mechanically pruned 1 to 2 days prior to infiltration. Chemical pruning plants 3, or 7 days prior to infiltration was also found to produce increased protein accumulation over non-pruned plants.

As shown in FIG. 9, increased levels of expression following infiltration of an antibody (935, FIG. 1) are observed when the coding region of interest is driven by the photosynthetic promoter plastocyanin when combined with pruning (mechanical pruning 12 hours prior to vacuum infiltration) and co-expression with a suppresser of silencing. Co-expression of 935 with the suppressor of silencing HcPro following pruning resulted in increased antibody accumulation levels 3-8 fold, compared to these observed in the absence of HcPro. When co-expressed with HcPro, plastocyanin- controlled expression reached average values of 280 mg/kg FW following pruning.

### Example 7

### Characterization of the Antibody Produced

Protein blot analyses (see Example 3) were used to reveal the level of assembly and fragmentation of the C5-1 IgG in plants producing the secreted (R612) and ER-retained (R610) forms of the protein, following both syringe- and vacuum-infiltration experiments. A Western blot probed with a H+L peroxidase-conjugated goat anti-mouse IgG was first used to highlight the presence of a maximum of antibody fragments independently of their origin on the C5-1 molecule. As shown in FIG. 3A, all protein extracts contained fragments of similar molecular sizes and in similar relative abundance, irrespective of the subcellular targeting strategy or infiltration method used. In each case, a major band (>85%) corresponding to the complete antibody at about 150 kDa, was revealed, with two minor bands at about135 kDa and about 100 kDa, showing that the antibody accumulated mainly in its fully assembled form (H₂L₂). Interestingly, fragments of similar electrophoretic mobility were also present in the control IgG1 purified from a murine tumor cell line (MOPC-21; Sigma #M9269), suggesting that the fragmentations produced in plants and mammalian cell lines were similar and probably resulted from common proteolytic activities. Similar results were also obtained with an anti-mouse heavy chain specific antibody for the detection.

To test the identity of the antibody fragments present in the extracts, an activity blot was used, in which the blotted proteins are probed with a peroxidase-conjugated human IgG1, the antigen of C5-1. The identity of a fully-assembled antibody of about150 kDa and can be seen in FIG. 3B. Furthermore, the fragmentation pattern observed in the Western blots, with the exception of a 100 kDa band (see FIG. 3A) are visible on the activity blot (FIG. 3B). Without wishing to be bound by theory, this result suggests that the 100 kDA fragment does not contain the Fab regions of the C5-1 antibody, and may consist, at least in part, of dimers of heavy chains, an intermediate of antibody assembly.

### Example 8

### Antibody Purification and Characterisation of the Purified Product

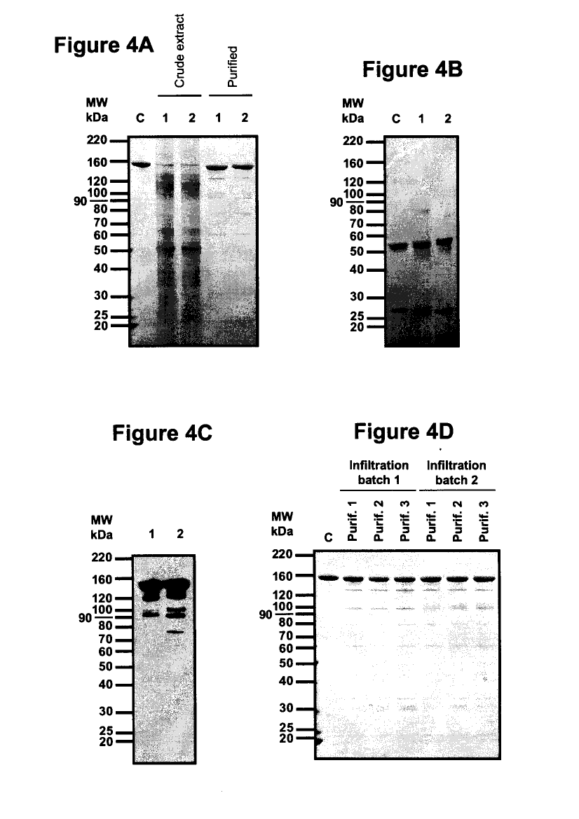

The antibody was purified from the biomass using a single Protein G affinity chromatographic step and the product obtained was analyzed by SDS-PAGE (see Example 4). The Coomassie stained gel presented in FIG. 4a shows a major band at 150 kDa in the eluate fraction from the Protein G. This band represents more than 85% of the purified product in both the secreted and ER-retained forms, and the contents in contaminants are identical for both forms (FIG. 4A, lanes 4 and 5). A Western blot analysis, probed with a polyclonal anti-mouse IgG, has shown the murine IgG origin of the major contaminant in the purified C5-1 fractions (data not shown). Under reducing conditions, two major products were detected at about 26 kDa and about 55 kDa which corresponds to the molecular weight of light and heavy chains, respectively (FIG. 4B, lane 2). The heavy chain of the ER-retained antibody showed a higher electrophoretic mobility than the heavy chain of the apoplastic antibody (FIG. 4B, lane 3) which is interpreted as the combined results of additional KDEL amino acids being present at the C-terminus and of differences in N-glycosylation due to the retention in the ER. FIG. 4C shows that the purified antibodies (150 kDa) bound to human IgG1, as did contaminating fragments of 75, 90, 100, and 120 kDa , highlighting the presence of at least one Fab segment in these fragments. The presence of Fab in the 100 kDa fragment contrasted with the result obtained from crude extract analysis, where the 100 kDa band did not bind to human IgG. It is hypothesized that either the amount of Fab-containing fragments migrating at 100 kDa in the crude extract was too low for detection with this activity blot or that the fragment migrating at 100 kDa consisted of two different molecules, one being heavy chain dimers (without Fab) and the other containing antigen-binding regions.

The reproducibility of this system for antibody production was assessed with a side-by-side comparison of purified products from 2 different infiltration batches and 3 distinct purification lots from each batch. The Coomassie-stained SDS-PAGE analysis of the purification lots showed the presence of identical bands in all lots, and in highly similar relative abundance (FIG. 4D).

### Example 9

### Modification of Antibody N-Glycosylation by Co-Expression of a Human Glactosyltransferase

To investigate whether transient co-expression could be used to control glycosylation of nascent proteins during transient expression, 35S-based expression cassettes comprising the native human β1,4galactosyltransferase (GalT) were prepared. R622 comprised GalT (FIG. 5B), and R621 comprised GalT catalytic domain fused to the CTS domain of N-acetylglucosaminyl transferase (GNTI; GNT1GalT (FIG. 5A). The CTS domain of N-acetylglucosaminyl transferase (GNTI) was selected as membrane anchorage for human GalT catalytic domain as GNT1 acts at an early stage of complex N-glycan synthesis in the ER and the cis-Golgi apparatus (Saint-Jore-Dupas et al., 2006). Without wishing to be limited by theory, sequestering GalT activity at an early stage of protein maturation may result in addition of β1,4galatose on maturating glycans and efficient inhibition of fucosylation and xylosylation of the core. These constructs were co-infiltrated in plants with C5-1.

Nicotiana benthamiana plant were infiltrated (see Example 2) with R612 (secreted for of C5-1), R612+R621(GNT1GalT) or R612+R622 (GalT) in the presence of HcPro. FIG. 6 shows an immunological analysis of C5-1 purified from these biomass samples.

Galactosylation of the antibody was estimated by affinodetection with the Erythrina cristagali agglutinin (ECA) which specifically binds β1,4galactose. As expected, no galactose was detected when C5-1 was expressed alone (R612; FIG. 6). Galactosylation was observed in C5-1 purified from co-infiltrations with R512+R622 (GalT) but not from co-infiltrations with R612+R621 (GNT1GalT, FIG. 6). Western blot performed using anti-α1,3fucose antibodies revealed fucosylation of the N-glycan on the control C5-1 expressed without galactosyltransferase. Fucosylation of the N-glycan was not detected on the antibody co-expressed with GNTIGalT, irrespectively of the agroinfiltration method, whereas co-expression with the native GalT did not lead to detectable reduction in fucosylation of the antibody (FIG. 6). Similar results were obtained with anti-β1,2xylose specific antibodies which showed the complete absence of xylose-specific immunosignals on C5-1 co-expressed with GNTIGalT and their presence when C5-1 was co-expressed with GalT (FIG. 6).

Coomassie stained gels for direct visual estimates of fully-assembled IgG, and Western and activity blots were performed on the same extracts. Based on this data, the antibody expression system as described reaches yields of 1.5 g/kg fresh weight with over 85% of the product consisting of full-size tetrameric IgG of about 150 kDa in crude extracts.

The addition of a KDEL peptide at the C-terminal of the heavy chain has been used previously to increase antibody accumulation (2-10×) by mediating the retrieval of the antibody from the Golgi back to the ER (Schillberg et al., 2003). With the expression system described herein, the addition of a KDEL peptide to the heavy chain of C5-1 doubled the yield of C5-1 when the HcPro suppressor of silencing was not used. The difference between the yield of C5-1 in the presence or absence of KDEL was significantly reduced when HcPro was used to reduce silencing. ER-retention did not influence product quality since the fragments observed in the crude extracts from plants producing the ER-retained and secreted forms of the antibody were identical in size and relative abundance.

All citations are hereby incorporated by reference.

The present invention has been described with regard to one or more embodiments. However, it will be apparent to persons skilled in the art that a number of variations and modifications can be made without departing from the scope of the invention as defined in the claims.

### REFERENCES

Bakker, H. et al. An antibody produced in tobacco expressing a hybrid beta-1,4-galactosyltransferase is essentially devoid of plant carbohydrate epitopes. Proc. Natl. Acad. Sci. U.S.A. 103, 7577-7582 (2006).

Boisson, M, et al. Arabidopsis glucosidase I mutants reveal a critical role of N-glycan trimming in seed development. EMBO J. 20, 1010-1019 (2001).

Brigneti, G. et al. Viral pathogenicity determinants are suppressors of transgene silencing in Nicotiana benthamiana. EMBO J. 17, 6739-6746 (1998).

Chua, Y. L., Watson, L. A., & Gray, J. C. The transcriptional enhancer of the pea plastocyanin gene associates with the nuclear matrix and regulates gene expression through histone acetylation. Plant Cell 15, 1468-1479 (2003).

Cox, K. M., et al. Glycan optimization of a human monoclonal antibody in the aquatic plant Lemna minor. Nat. Biotechnol. 24, 1591-1597 (2006).

D'Aoust, M. A. et al. Efficient and reliable production of pharmaceuticals in alfalfa. Molecular Farming, pp 1-12. Rainer Fischer and Stefan Schillberg (eds.), Wiley-VCH, Weinheim, Germany (2004).

Darveau, A., Pelletier, A. & Perreault, J. PCR-mediated synthesis of chimeric molecules. Methods Neurosc. 26, 77-85 (1995).

Elmayan, T. & Vaucheret, H. Expression of single copies of a strongly expressed 35S transgene can be silenced post-transcriptionally. Plant J. 9, 787-797 (1996).

Fischer, R. et al. Towards molecular farming in the future: transient protein expression in plants. Biotechnol. Appl. Biochem. 30, 113-116 (1999a).

Fischer, R., Drossard, J., Commandeur, U., Schillberg, S. & Emans, N. Towards molecular farming in the future: moving from diagnostic protein and antibody production in microbes to plants. Biotechnol. Appl. Biochem. 30, 101-108 (1999b).

Giritch, A. et al. Rapid high-yield expression of full-size IgG antibodies in plants coinfected with noncompeting viral vectors. Proc. Natl. Acad. Sci. U.S.A. 103, 14701-14706 (2006).

Gomord, V., Chamberlain, P., Jefferis, R. & Faye, L. Biopharmaceutical production in plants: problems, solutions and opportunities. Trends Biotechnol. 23, 559-565 (2005).

Hamilton, A., Voinnet, O., Chappell, L. & Baulcombe, D. Two classes of short interfering RNA in RNA silencing. EMBO J. 21, 4671-4679 (2002).

Hiatt, A., Cafferkey, R. & Bowdish, K. Production of antibodies in transgenic plants. Nature 342, 76-78 (1989).

Hiatt, A. & Pauly, M. Monoclonal antibodies from plants: a new speed record. Proc. Natl. Acad. Sci. U.S.A. 103, 14645-14646 (2006).

Hibino, Y., Ohzeki, H., Sugano, N. & Hiraga, K. Transcription modulation by a rat nuclear scaffold protein, P130, and a rat highly repetitive DNA component or various types of animal and plant matrix or scaffold attachment regions. Biochem. Biophys. Res. Commun. 279, 282-287 (2000).

Höfgen, R. & Willmitzer, L. Storage of competent cells for Agrobacterium transformation. Nucleic Acid Res. 16, 9877 (1988).

Hull, A. K. et al. Human-derived, plant-produced monoclonal antibody for the treatment of anthrax. Vaccine 23, 2082-2086 (2005).

Kapila, J., De Rycke, R., Van Montagu, M. & Angenon, G. An Agrobacterium-mediated transient gene expression system for intact leaves. Plant Sci. 122, 101-108 (1997).

Kathuria, S. R. et al. Functional recombinant antibodies against human chorionic gonadotropin expressed in plants. Curr. Sci. 82, 1452-1456 (2002).

Ko, K. et al. Function and glycosylation of plant-derived antiviral monoclonal antibody. Proc. Natl. Acad. Sci. U.S.A. 100, 8013-8018 (2003).

Ko, K. & Koprowski, H. Plant biopharming of monoclonal antibodies. Virus Res. 111, 93-100 (2005).

Koprivova, A. et al. Targeted knockouts of physcomitrella lacking plant-specific immunogenic N-glycans. Plant Biotechnol. J. 2, 517-523 (2004).

Khoudi, H. et al. Production of a diagnostic monoclonal antibody in perennial alfalfa plants. Biotechnol. Bioeng. 64, 135-143 (1999).

Leutwiler, L. S., Meyerowitz, E. M., Tobin, E. M. Structure and expresision of three light harvesting chrlorphyll a/b binding protein genes in Arabidopsis thaliana. Nuc. Acids Res. 10, 4051-4064 (1986).

Liu, L & Lomonossoff, G. P. Agroinfection as a rapid method for propagating Cowpea mosaic virus-based constructs. J. Virol. Methods 105, 343-348 (2002).

Ma, J. K-C., Drake, P. M. W., Chargelegue, D., Obregon, P. & Prada, A. Antibody processing and engineering in plants, and new strategies for vaccine production. Vaccine 23, 1814-1818 (2005).

Misaki, R., Fujiyama, K. & Seki, T. Expression of human CMP-N-acetylneuraminic acid synthetase and CMP-sialic acid transporter in tobacco suspension-cultured cell. Biochem. Biophys. Res Com. 339, 1184-1189 (2004).

Paccalet et al. Engineering of a sialic acid synthesis pathway in transgenic plants by expression of bacterial Neu5Ac-synthesizing enzymes. Plant Biotech. J. 5, (2007).

Pagny, S. et al. Structural requirements for Arabidopsis β1,2-xylosyltransferase activity and targeting to the Golgi. Plant J. 33, 189-203 (2003).

Peterson, E., Owens, S. M. & Henry, R. L. Monoclonal antibody form and function: manufacturing the right antibodies for treating drug abuse. AAPS J. 8, E383-E390 (2006).

Petrucelli, S. et al. A KDEL-tagged monoclonal antibody is efficiently retained in the endoplasmic reticulum in leaves, but is both partially secreted and sorted to protein storage vacuoles in seeds. Plant Biotechnol. J. 4, 511-527 (2006).

Pwee, K-H. & Gray, J. C. The pea plastocyanin promoter directs cell-specific but not full light-regulated expression in transgenic tobacco plants. Plant J. 3, 437-449 (1993).

Rodriguez, M. et al. Transient expression in tobacco leaves of an aglycosylated recombinant antibody against the epidermal growth factor receptor. Biotechnol. Bioeng. 89, 188-194 (2005).

Saint-Jore-Dupas, C. et al. Plant N-glycan processing enzymes employ different targeting mechanisms for their spatial arrangement along the secretory pathway. Plant Cell 18, 3182-3200 (2006).

Sambrook J., and Russell, D. W., Molecular Cloning: A Labaratory Manual. Cold Spring Harbor Laboratory Press, Cold Spring Harbor, N.Y. (2001).

Sandhu, J. S., Webster, C. I., & Gray, J. C. A/T-rich sequences act as quantitative enhancers of gene expression in transgenic tobacco and potato plants. Plant Mol. Biol. 37, 885-896 (1998).

Schillberg, S., Fischer, R. & Emans, N. Molecular farming of recombinant antibodies in plants. Cell. Mol. Life Sci. 60, 433-445 (2003).

Sharp, J. M. & Doran, P. M. Characterization of monoclonal antibody fragments produced by plant cells. Biotechnol. Bioeng. 73, 338-346 (2001).

Sriraman, R. et al. Recombinant anti-hCG antibodies retained in the endoplasmic reticulum of transformed plants lack core-xylose and core-α(1-3)-fucose residues. Plant Biotechnol. J. 2, 279-287 (2004).

Stockhaus, J., Schell, J., Willmitzer, L. Correlation of the expression of the nuclear photosynthetic gene ST-LS 1 with the presence of chloroplasts. EMBO J. 8, 2445-2451 (1989)

Strasser, R., Altmann, F., Mach, L., Glössl, J. & Steinkellner, H. Generation of Arabidopsis thaliana plants with complex N-glycans lacking β31,2 linked xylose and core α1,3-linked fucose. FEBS Lett. 561, 132-136 (2004).

Szittya, G. et al. Low temperature inhibits RNA silencing-mediated defence by the control of siRNA generation. EMBO J. 22, 633-640 (2003).

Verch, T, Yusibov, V. & Koprowski, H. Expression and assembly of a full-length monoclonal antibody in plants using a plant virus vector. J. Immunol. Methods 220, 69-75 (1998).

Watzele, G., Bachofner, R. & Berger, E. G. Immunocytochemical localization of the Golgi apparatus using protein-specific antibodies to galactosyltransferase. Eur. J. Cell. Biol. 56, 451-458 (1991).

Yusibov, V., Rabindran, S., Commandeur, U. Twyman, R. M. & Fischer, R. The potential of plant virus vectors for vaccine production. Drugs R.D. 7, 203-217 (2006).

1-20. (canceled)

21. A method for synthesizing a protein of interest within a plant or a portion of a plant comprising,
i) pruning the plant or portion of the plant to produce a pruned plant or portion of the plant, the pruning consists of:
(a) removing, killing, inducing necrosis or reducing growth of one or more than one axillary bud, one or more than one apical bud, or both one or more than one axillary bud and one or more than one apical bud; or
(b) applying a chemical compound that reduces apical dominance;
ii) introducing, before or after the step of pruning, one or more than one nucleic acid sequence encoding a protein of interest operatively linked with a regulatory region that is active in the plant, into the pruned plant or portion of the plant, in a transient manner and
iii) maintaining the pruned plant or portion of the plant under conditions that permit the nucleic acid sequence encoding the protein of interest to be episomally transcribed and the protein of interest to be expressed in the plant or a portion of the plant.

22. The method of claim 21, wherein in the step of introducing (step ii), two or more than two nucleic acid sequences are introduced within the plant.

23. The method of claim 21, wherein in the step of introducing (step ii), the one or more than one nucleic acid sequence is introduced into the pruned plant or portion of the plant under vacuum.

24. The method of claim 21, wherein in the step of introducing (step ii), the one or more than one nucleic acid sequence is introduced into the pruned plant or portion of the plant using syringe inflitration.

25. The method of claim 21, wherein in the step of pruning (step i) the pruning consists of removing, killing, inducing necrosis or reducing growth of one or more than one axillary bud, one or more than one apical bud, or both one or more than one axillary bud and one or more than one apical bud.

26. The method of claim 21, wherein in the step of pruning (step i) the pruning consists of applying a chemical compound that reduces apical dominance.

27. The method of claim 21, wherein in the step of introducing (step ii), the regulatory region is a promoter obtained from a photosynthetic gene.

28. The method of claim 27, wherein the regulatory region is a plastocyanin promoter, a plastocyanin 3′UTR and terminator, or both.

29. The method of claim 22, wherein one of the two or more than two nucleic acid sequences encodes a suppressor of silencing.

30. The method of claim 29, wherein the suppressor of silencing is HcPro.

31. The method of claim 21, wherein the protein of interest is an antibody, an antigen or a vaccine.

32. A method for synthesizing a protein of interest within a plant or a portion of a plant comprising,
i) pruning the plant or portion of the plant to produce a pruned plant or portion of the plant, the pruning consists of:
(a) removing, killing, inducing necrosis or reducing growth of one or more than one axillary bud, one or more than one apical bud, or both one or more than one axillary bud and one or more than one apical bud; and/or
(b) applying a chemical compound that reduces apical dominance;
ii) introducing, before or after the step of pruning, one or more than one nucleic acid sequence encoding a protein of interest operatively linked with a regulatory region obtained from a photosynthetic gene that is active in the plant or portion of the plant, in a transient manner, and
iii) maintaining the pruned plant or portion of the plant under conditions that permit the nucleic acid sequence encoding the protein of interest to be episomally transcribed and the protein of interest to be expressed in the plant or a portion of the plant.
